SEQUENCE LISTING (8 sequences)

SEQ ID NO: 1 - RNA, 41 nt, synthetic construct
  source 1..41: mol_type = other RNA
taatttctactaagtgtagatgtggtattcttgctagttac

SEQ ID NO: 2 - DNA, 20 bp, synthetic construct
  source 1..20: mol_type = other DNA
gtaactagcaagaataccac

SEQ ID NO: 3 - protein, 8 aa, synthetic construct
TTATTATT

SEQ ID NO: 4 - RNA, 20 nt, synthetic construct
  source 1..20: mol_type = other RNA
gtggtattcttgctagttac

SEQ ID NO: 5 - RNA, 20 nt, synthetic construct
  source 1..20: mol_type = other RNA
ttctcttaattccttgatag

SEQ ID NO: 6 - DNA, 31 bp, synthetic construct
  source 1..31: mol_type = other DNA
ctatcaaggaattaagagaagcaacatctcc

SEQ ID NO: 7 - DNA, 31 bp, synthetic construct
  source 1..31: mol_type = other DNA
gtcccgtcgctatcaagcaaccaacatctcc

SEQ ID NO: 8 - RNA, 20 nt, synthetic construct
  source 1..20: mol_type = other RNA
ggagatgttggttgcttgat

(19) United States
(12) Patent Application Publication
(10) Pub. No.: US 2025/0250616 A1
(43) Pub. Date: Aug. 7, 2025

(54) ENZYME KINETICS ANALYSES OF CRISPR ENDONUCLEASES

(71) Applicant: The Board of Trustees of the Leland Stanford Junior University, Stanford, CA (US)

(72) Inventors: Juan Santiago, Stanford, CA (US); Alexandre Avaro, Stanford, CA (US); Ashwin Ramachandran, Stanford, CA (US); Diego Huyke, Stanford, CA (US); Charles Blanluet, Stanford, CA (US)

(73) Assignee: The Board of Trustees of the Leland Stanford Junior University, Stanford, CA (US)

(21) Appl. No.: 18/855,157

(22) PCT Filed: Apr. 12, 2023
(86) PCT No.: PCT/US2023/065690

§ 371 Date: Oct. 8, 2024

Related U.S. Application Data
(60) Provisional application No. 63/330,764, filed on Apr. 13, 2022.

(51) Int. Cl. C12Q 1/6823; C12Q 1/683

(52) U.S. Cl. CPC C12Q 1/6823; C12Q 1/683

(57) ABSTRACT

Provided are methods of analyzing target nucleic acids, the methods comprising, using the target nucleic acids as reagents and reporters as substrates, assaying enzyme kinetic parameters of Clustered Regularly Interspaced Short Palindromic Repeats (CRISPR) endonucleases comprising guide RNAs (gRNAs) that hybridize with reference nucleic acids. In certain aspects, certain such methods comprise comparing a sequence of a target nucleic acid with a sequence of a reference nucleic acid. In some embodiments, such comparison is based on the rates of cleavage of the reporters at plurality of concentrations of the reporters. The kinetic parameters can also be the Michaelis-Menten constant (K_{M}), the apparent turnover rate (K*_{cat}), and/or the apparent catalytic efficiency (K*_{cat}/K_{M}). Kits for performing the methods of the disclosure are also provided.

### CROSS-REFERENCE TO RELATED APPLICATIONS

This application claims priority to U.S. Provisional Application No. 63/330,764 (filed Apr. 13, 2022), the disclosure of which is incorporated herein by reference in its entirety.

### SEQUENCE LISTING

A Sequence Listing is provided herewith as a Sequence Listing XML, “STAN-2084WO_SEQ_LIST” created on Apr. 11, 2023, and having a size of 1500 bytes. The contents of the Sequence Listing XML are incorporated by reference herein in their entirety.

### INTRODUCTION

Clustered Regularly Interspaced Short Palindromic Repeats (CRISPR) technology is specific and configurable. These features led to commercial interest in the development of CRISPR-based molecular diagnostics. As depicted in FIG. 1, CRISPR-based diagnostic assays use a CRISPR-endonuclease, such as a CRISPR-associated (Cas) enzyme, complexed with a synthetic guide RNA (gRNA) containing an enzyme-specific region (shown as a hairpin structure in the figure) and a second, reconfigurable region complementary to a target nucleic acid. The assay involves two steps. The first step is a cis-cleavage reaction where the enzyme recognizes and cleaves a specific target nucleic acid, activating the enzyme. The second step is a non-specific trans-cleavage reaction where the activated enzyme indiscriminately cleaves other nucleic acids. Typically in assays, such other nucleic acids are synthetic ssDNA/ssRNA reporters with fluorophore-quencher pairs.

### SUMMARY

In certain aspects, the disclosure provides CRISPR-based detection of target nucleic acids, such as CRISPR-based molecular diagnostics. The methods disclosed herein facilitate detection of even minor variations in nucleic acid sequences, including SNPs.

In some cases, the CRISPR endonucleases used in the methods disclosed herein comprise a type V CRISPR/Cas proteins (e.g., Cas 12 proteins such as Cpf1 (Cas12a) and C2c1 (Cas12b)), which can promiscuously cleave non-targeted single stranded DNA (ssDNA) once activated by detection of a target nucleic acid.

According to certain aspects, the disclosure provides methods of analyzing target nucleic acids, the methods comprising, using the target nucleic acids as reagent and reporters as a substrate, assaying an enzyme kinetic parameter of a CRISPR endonuclease comprising a guide RNA (gRNA) that hybridizes with a reference nucleic acid. The CRISPR endonuclease can be a type V CRISPR/Cas endonuclease.

The reporter can be a single or double stranded nucleic acid, which, when cleaved by the CRISPR endonuclease, emits a detectable signal, such as a fluorescent signal.

In some cases, the enzyme kinetic parameter is the rates of cleavage of the reporter at different concentrations of the reporter. In certain such cases, the enzyme kinetic parameter is the initial rates of cleavage of the reporter at different concentrations of the reporter.

In some cases, the enzyme kinetic parameter can be the Michaelis-Menten constant (K_{M}), apparent enzyme turnover rate (k*_{cat}), apparent catalytic efficiency (k*_{cat}/K_{M}), enzyme turnover rate (k_{cat}), or catalytic efficiency (k_{cat}/K_{M}) of the CRISPR endonuclease.

In some aspects, the disclosure provides methods of comparing a sequence of a target nucleic acid with a sequence of a reference nucleic acid, comprising contacting the target nucleic acid as a reagent and a plurality of concentrations of a reporter as a substrate with a Clustered Regularly Interspaced Short Palindromic Repeats (CRISPR) endonuclease comprising a guide RNA (gRNA) that hybridizes with the reference nucleic acid, and quantifying the rates of cleavage of the reporter at the plurality of concentrations of the reporter.

Depending on the assayed enzyme kinetic parameter as compared to enzyme kinetic parameter of the reference nucleic acid, the target nucleic acid is identified as having the same sequence or a different sequence than the reference nucleic acid.

Further aspects of the invention provide a kit for performing the methods disclosed herein. Certain such kits comprise a reporter; a CRISPR endonuclease comprising a guide RNA comprising a sequence that is perfectly complementary to a reference nucleic acid; and a non-transitory computer readable media comprising instructions, which when executed by a processor, cause the processor to analyze the results of the assay of the CRISPR endonuclease using a target nucleic acid as a reagent and the reporter as a substrate and provide an enzyme kinetic parameter of the CRISPR endonuclease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Reference to color in the drawings refers to color drawings, which may be provided based on the jurisdiction.

As labeled in the Figures, the term “substrate” refers to the “reporter” and the term “target” refers to the analyzed target nucleic acid.

FIG. 1. Michaelis-Menten kinetics model of trans-cleavage of activated CRISPR-Cas12. Cas12 (gray blob) is functionalized with a synthetic guide RNA (shown as a hairpin structure). In this rate-limiting trans-cleavage step, the enzyme has been functionalized by recognition and cleavage of the target DNA molecule (cleaved ssDNA target shown as a small blue strand). The enzyme indiscriminately cleaves ssDNA. By design, stoichiometry then favors cleaving of nucleic acid reporter probes functionalized with a fluorophore (F) and quencher (Q). K_{M} is the Michaelis-Menten constant defined in terms of the off-rate (kᵣ), apparent turnover rate (k*_{cat}), and forward rate (kf).

FIGS. 2A-2D. Schematic of CRISP-Cas12a detection and preliminary experimental results based on endpoint fluorescence detection. A. Schematic representation of a Cas12-gRNA complex which activates upon recognition of a ssDNA target and begins trans- (or collateral) cleavage of fluorescently labeled ssDNA reporters. The schematic within the dashed box depicts the interaction between the gRNA (SEQ ID NO: 4) and WT target (SEQ ID NO: 2). B. Fluorescence versus time for WT and SNP targets. Cas12-gRNA complex and reporter concentrations were respectively 2 and 800 nM. C. Fluorescence of WT and SNP targets at 10 min (left column) and 45 min (right column). Data has been normalized to the highest value of each instance. Asterisks denote the grids which correspond to WT. The enzyme and reporter concentrations match those in B. C9T is brighter than WT at 10 min. D. WT fluorescence signal minus that of SNP versus time for SNPs at position #9. The Cas12-gRNA complex and reporter concentrations were respectively 10 and 800 nM.

FIGS. 3A-3C. Michaelis-Menten experiments of SNPs. A. Fluorescence versus time curves show trans-cleavage of fluorescently labeled ssDNA reporters by a Cas12-gRNA complex activated by WT or SNPs in nucleotide position #4. B. Cleavage rate versus initial substrate (uncleaved reporter) concentration for WT and SNPs at position #3. The Cas12-gRNA complex concentration in FIGS. 3A and 3B was 2 nM. C. Heatmap of apparent turnover rate k*_{cat} for WT and all associated SNPs, estimated from fits to the Michaelis-Menten curve. Asterisks in the heatmap denote the grids which correspond to WT.

FIG. 4. WT differentiation based on K_{M} m. Heatmap of K_{M} for WT and all associated SNPs as estimated from fits to the Michaelis-Menten curve. Heat map is arranged so that lighter shades indicate high affinity (low K_{M}). Asterisks denote the cells corresponding to WT. All but one of 60 SNPs (C17A) exhibited greater K_{M} (lower affinity) than for WT. Thus, K_{M} provides a time- and target concentration-insensitive technique for differentiating WT from SNPs using CRISPR-based assays.

FIGS. 5A-5D. Implications of Michaelis-Menten analysis for CRISPR-based detection of SNPs. A. Measured apparent catalytic efficiency (k*_{cat}/K_{M}) for WT and all associated SNPs, as determined from fits to the Michaelis-Menten curves. Asterisks denote the cells which correspond to WT. For all cases, the k*_{cat}/K_{M} of the WT was consistently larger than all SNPs. B. Normalized difference to the WT endpoint fluorescence after 45 min (in black) or apparent catalytic efficiency k*_{cat}/K_{M} (in gray). Each row of plots corresponds to mutations to the base indicated in the ordinate. Higher values correspond to a greater ability to differentiate among WT and SNP targets. k*_{cat}/K_{M} was a better differentiator of WT versus SNP than endpoint fluorescence for 54 SNPs and worse only for T15C, C17A, C17G, and C18G. C and D. CRISPR kinetic activity analysis of this disclosure outperforms end-point signal-based detection. Even 2× variation in the target concentration can cause inaccuracies in the fluorescence signal detection based assay.

FIGS. 6A-6C. Predicted scenarios wherein endpoint fluorescence could not differentiate WT from SNP targets. A. Table of contrived kinetic parameters input into the experimentally validated Michaelis-Menten model. B. Predicted Trans-cleavage progress curves of cleaved reporter, i.e., substrate concentration versus time. C. Normalized difference to the WT (analogous to Eq. (1)) versus time. Generated from the same dataset in B. The datasets which begin above the lines defined by a value of zero in the ordinate correspond to the conditions where k*_{cat} was increased while E0 was proportionally decreased. Conversely, the datasets which begin below this line correspond to the conditions where K_{M} was decreased while E0 was proportionally increased. Note all differences approach zero after about 1.5 h, as expected.

FIGS. 7A-7B. Proposed CRISPR assays for high specificity. A. Assay 1 achieves very high specificity by measuring K_{M} for CRISPR based detection and does not require knowledge of sample concentration. B. Assay 2 requires an amplification step (and perhaps a quantification step) but maximizes specificity by extracting both k_{cat}/K_{M} and K_{M}. Assay 2 offers higher sensitivity (since it always includes pre-amplification) and the maximum specificity (since both k*_{cat}/K_{M} and K_{M} are quantified.

FIG. 8. SNP C9T has a high trans-activated turnover rate. Difference to WT fluorescence signal (normalized by WT signal) versus time for various trans-activated concentrations of C9T. Plot was generated in MATLAB (R2021b, Mathworks, USA) using an experimentally validated numerical model of Michaelis-Menten kinetics. The enzyme kinetic parameters (k_{cat} and K_{M}) for the WT and C9T were extracted from Michaelis-Menten curves for those same targets (Table 1). The WT-activated enzyme and initial substrate concentration were respectively 10 and 800 nM (the same experimental conditions as FIG. 2D). The low SNP concentration conditions (6 and 7 nM) resulted in substrate cleavage initially faster and then slower than WT. All conditions resulted in no difference to WT (when all substrate was fully cleaved) for times greater than 80 min.

FIG. 9. DNA reporter calibration curves. The figure shows the calibration curves for the cleaved and uncleaved ssDNA reporters. The calibration curves were used to quantify cleavage activity in molar units. Measured fluorescence versus concentration of uncleaved and cleaved FAM-BHQ DNA reporters. Lines (dashed line) and power law curves (solid lines) of best fit to the experimental data were obtained by linear regression. Data was taken in a 7500 Fast Real-Time PCR system (Applied Biosystems, CA, USA). Significant inner filter effect leads to the non-linearity and this is accounted for by the exponential law.

FIGS. 10A-10I. Michaelis-Menten fits for all SNPs. This figure presents Michaelis-Menten fits to the initial reaction for all SNPs at all positions. These data comprise measurements across 60 SNPs over 20 nucleotide positions, i.e., LbCas12 Michaelis-Menten fits for all SNPs in nucleotide positions #1-9. A. Cleavage rate versus initial substrate (uncleaved reporter) concentration for WT and SNPs at position #1. Shown together with the data are fits to the Michaelis-Menten equation. B-I. Same data as A for WT and, respectively, SNPs in positions #2-9. The Cas12-gRNA complex concentration for all experiments was 2 nM.

FIGS. 11A-11I. LhCas12 Michaelis-Menten fits for all SNPs in nucleotide positions #10-18. A. Cleavage rate versus initial substrate (uncleaved reporter) concentration for WT and SNPs at position #10. Also shown are fits to the Michaelis-Menten equation. B-I. Same data as A for WT and, respectively, SNPs in positions #11-18. The Cas12-gRNA complex concentration for all experiments was 2 nM.

FIGS. 12A-12B. LbCas12 Michaelis-Menten fits for all SNPs in nucleotide positions #19 and 20. A. Cleavage rate versus initial substrate (uncleaved reporter) concentration for WT and SNPs at position #19. Also shown are fits to the Michaelis-Menten equation. B. Same data as a for WT and SNPs in position #20. The Cas12-gRNA complex concentration for all experiments was 2 nM.

FIG. 13. An example of a workflow for genotyping for a tumor marker with CRISPR endonuclease based kinetics assay disclosed herein.

FIG. 14. Assay development and assay components and their influence on CRISPR endonuclease kinetics (from top to bottom SEQ ID NOs: 5-8).

FIG. 15. Synthetic sensitivity study for ssDNA and dsDNA.

FIG. 16. An exemplary application of the CRISPR endonuclease kinetic assays disclosed herein for tumor genotyping. (MM=Michaelis Menten.)

FIG. 17. An exemplary assay when a mixture of WT and mutant target nucleic acid may be present in a sample.

FIGS. 18A-18B. Cleavage rates at different concentrations of the target nucleic acids (as single stranded or double stranded DNA) as well as reporters.

FIG. 19. Applications for highly specific detection are tumor genotyping at the lab's bench and early disease risk assessment at the clinic.

FIG. 20. Highly specific assays disclosed herein to detect hot-spot mutations in tumor at the labs bench, without NGS, using common PCR equipment.

FIG. 21. Highly specific and low equipment assay disclosed herein provide critical need for early screening of inherited diseases risks in clinical setting.

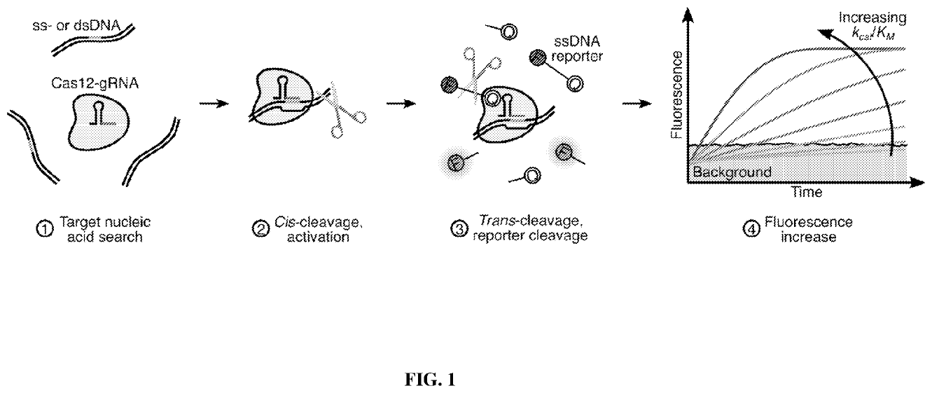

### DETAILED DESCRIPTION

Specificity in nucleic acid detection refers to the ability to differentiate among small variations of the nucleic acid sequences. The differentiation of SNPs are especially challenging. Certain aspects of the disclosure provide CRISPR-based detection of nucleic acids, for example, CRISPR-based molecular diagnostics. Such detections can be made very specific to small variations of nucleic acid sequences, including SNPs.

Conventionally, CRISPR-based nucleic acid detection primarily relies on end-point detection to differentiate sequences. Endpoint detection involves measuring fluorescence versus time for various targets and comparing the signals. In such assays, the difference in fluorescence is used to differentiate between a mutant versus a WT target. However, endpoint detection assays are fraught with inaccuracies and have limited applications.

To address the shortcomings of such assays, certain aspects of the disclosure provide methods of analyzing target nucleic acids, the methods comprising, using the target nucleic acids as a reagent and reporters as a substrate, assaying certain enzyme kinetic parameters of CRISPR endonucleases comprising gRNA that hybridize with the target nucleic acids. Thus, the methods of the disclosure involve taking additional measurements at multiple dilutions of the reporters to obtain CRISPR endonuclease activity at different concentrations of the reporters. Such information can be then used to measure the apparent turnover rate (k*_{cat}) and Michaelis-Menten constant (K_{M}) for any one target.

These kinetic parameters are a fundamental property of the enzyme and substrate. Therefore, these kinetic parameters provide a more specific measurement of the enzyme-substrate interactions.

Advantageously, measuring K_{M} according to this disclosure can be performed even when the target concentration is unknown. This enables identifying some mutations even without knowing the concentration of a target in a sample. Also, for applications where some estimate of the target concentration is available, the ratio k_{cat}/K_{M} can differentiate between WT and mutants.

Before the present invention is described in greater detail, it is to be understood that this invention is not limited to embodiments described, as such may vary. It is also to be understood that the terminology used herein is for the purpose of describing embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As used herein and in the appended claims, the singular forms “a,” “an,” and “the” include plural referents unless the context clearly dictates otherwise. For example, reference to “a protein” includes a plurality of such proteins and reference to “a mutation” includes reference to one or more discrete mutations, and so forth. It is further noted that the claims may be drafted to exclude any element, e.g., any optional element. As such, this statement is intended to serve as antecedent basis for use of such exclusive terminology as “solely,” “only,” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal,” or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “wild type” (WT) as used herein refers to an initially identified version of a nucleic acid or a version of the nucleic acid that is predominantly found in nature. A WT nucleic acid can naturally mutate to produce another naturally occurring mutant nucleic acid. Such mutant nucleic acid may become predominant in nature; however, it is not typically not referenced as a WT nucleic acid.

For example, the sequence of SARS-CoV-212 initially identified is referenced as “WT” and the later variants of this virus are called mutants or variants. Such later identified variants may become predominant; however, they are still considered mutants or variants because such comparison is made with respect to the initially identified virus.

The term “Single Nucleotide Polymorphism” (SNP) refers to differences in the nucleic acid sequences of an organism that are caused by point mutations. SNPs typically produce different alleles containing alternative bases at a given position. Most common or dominant versions of SNP can be referenced as “wild-type.” However, in many cases, one version of SNP at a particular position may not be dominant and several SNPs may be common.

A “target nucleic acid” as used herein is a polynucleotide (e.g., DNA such as genomic DNA) that includes a site (“target site” or “target sequence”) targeted by a modified CRISPR endonuclease of the present disclosure. The target sequence is the sequence to which the guide sequence of a guide nucleic acid (e.g., guide RNA; e.g., a dual guide RNA or a single-molecule guide RNA) will hybridize. Suitable hybridization conditions include physiological conditions normally present in a cell. For a double stranded target nucleic acid, the strand of the target nucleic acid that is complementary to and hybridizes with the guide RNA is referred to as the “complementary strand” or “target strand.” The strand of the target nucleic acid that is complementary to the “target strand” (and is therefore not complementary to the guide RNA) is referred to as the “non-target strand” or “non-complementary strand.”

The term “cleavage” refers to the breakage of the covalent backbone of a target nucleic acid (e.g., RNA, DNA). Both single-stranded cleavage and double-stranded cleavage are possible, and double-stranded cleavage can occur as a result of two distinct single-stranded cleavage events.

The term “endonuclease” refers to an enzyme that possesses catalytic activity for nucleic acid cleavage (e.g., ribonuclease activity (ribonucleic acid cleavage), deoxyribonuclease activity (deoxyribonucleic acid cleavage), etc.).

A gRNA and a CRISPR endonuclease form a complex (e.g., bind via non-covalent interactions). The guide RNA provides target specificity to the complex by including a guide sequence (a nucleotide sequence that is complementary to a sequence of a target nucleic acid). The CRISPR endonuclease of the complex provides the site-specific cleavage activity. In other words, the CRISPR endonuclease is guided to a target nucleic acid sequence (e.g. a target sequence in a chromosomal nucleic acid, e.g., a chromosome; a target sequence in an extrachromosomal nucleic acid, e.g. an episomal nucleic acid, a minicircle, an ssRNA, an ssDNA, etc.; a target sequence in a mitochondrial nucleic acid; a target sequence in a chloroplast nucleic acid; a target sequence in a plasmid; a target sequence in a viral nucleic acid; etc.) by virtue of its association with the guide RNA.

The “guide sequence” also referred to as the “targeting sequence” of a guide RNA can be modified so that the guide RNA can target a CRISPR endonuclease to any desired sequence of any desired target nucleic acid. Thus, for example, a guide RNA can have a targeting segment with a sequence (a guide sequence) that has complementarity with (e.g., can hybridize to) a sequence in a nucleic acid any organism.

In some cases, a guide RNA includes two separate nucleic acid molecules: an “activator” and a “targeter” and is referred to herein as a “dual guide RNA,” a “double-molecule guide RNA,” or a “two-molecule guide RNA,” a “dual guide RNA,”, or a “dgRNA.” In some embodiments, the activator and targeter are covalently linked to one another (e.g., via intervening nucleotides) and the guide RNA is referred to as a “single guide RNA,” a “Cas9 single guide RNA,” a “single-molecule Cas9 guide RNA,” or a “one-molecule Cas9 guide RNA,” or simply “sgRNA.”

Examples of various CRISPR endonucleases and guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; Shmakov et al., Nat Rev Microbiol. 2017 March; 15(3):169-182; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; each of which is hereby incorporated by reference in its entirety.

In some cases, a guide nucleic acid comprises ribonucleotides only, deoxyribonucleotides only, or a mixture of ribonucleotides and deoxyribonucleotides. In some cases, a guide nucleic acid comprises ribonucleotides only, and is referred to herein as a “guide RNA.” In some cases, a guide nucleic acid comprises deoxyribonucleotides only, and is referred to herein as a “guide DNA.” In some cases, a guide nucleic acid comprises both ribonucleotides and deoxyribonucleotides. A guide nucleic acid can comprise combinations of ribonucleotide bases, deoxyribonucleotide bases, nucleotide analogs, modified nucleotides, and the like; and may further include naturally-occurring backbone residues and/or linkages and/or non-naturally-occurring backbone residues and/or linkages.

An enzyme kinetic parameter can be any parameter that represents an enzyme kinetic property of a CRISPR endonuclease. Particularly, an enzyme kinetic parameter is any parameter that represents an enzyme kinetic property of a CRISPR endonuclease in cleaving a reporter.

For example, an enzyme kinetic parameter can be the rates of cleavage of the reporter at different concentrations of the reporter. An enzyme kinetic parameter can also be the initial rates of cleavage of the reporter at different concentrations of the reporter, such as, rates of cleavage of the reporter during initial 1 to 10 minutes, such as initial 1, 2, 3, 4, 5, 6, 7, 8, 9, or 10 minutes after initiation of the CRISPR endonuclease mediated cleavage of the reporter.

Michaelis-Menten constant (K_{M}) represents the affinity of an enzyme for its substrate. K_{M} is defined as the concentration of a substrate necessary to allow an enzyme to function at half of the enzyme's maximal velocity.

To characterize the underlying Michaelis-Menten kinetics of SNP-activated trans-cleavage activity, apparent turnover rate (k*_{cat}) can be measured. Apparent turnover rate is defined by interpreting the data as if all enzyme-target nucleic acid complexes are trans-activated by WT or SNP targets. That is, k*_{cat} is defined as the ratio of the maximum achievable reaction velocity divided by the target concentration. k*_{cat} (rather the underlying turnover rate K_{cat}) is reported because the fraction of activated enzyme-target nucleic acid is unknown for the certain targets, such as complexes of mutants. k*_{cat} provides the turnover rate for the enzyme considering that the enzyme may not be fully saturated with the substrate.

Apparent catalytic efficiency is calculated as the ratio of apparent turnover rate (k*_{cat}) to K_{M} i.e. k*_{cat}/K_{M}.

Turnover rate is defined as the number of substrate molecules converted into product by an enzyme molecule in a unit time when the enzyme is fully saturated with substrate.

Catalytic efficiency of an enzyme is the efficiency with which the enzyme converts a given substrate to a given product. Catalytic efficiency is calculated as the ratio of turnover rate (k_{cat}) to K_{M} i.e. k_{cat}/K_{M}.

The term “reporter” as used herein is a labeled polynucleotide that acts as a substrate for activated CRISPR endonuclease, particularly, activated type V CRISPR endonuclease. As noted above, an activated type V CRISPR endonuclease indiscriminately cleaves other nucleic acids. Such other nucleic acids can be ssDNA, ssRNA, dsDNA, or dsRNA reporters. In some cases, these nucleic acids are labeled with a detectable label, such as with fluorophore-quencher pairs so that the cleavage of the reporter is indicated by a detectable label, such as fluorescence. Many detectable labels, particularly fluorescent-quencher pairs are well-known in the art and use of such detectable labels is within the purview of the invention.

Fluorescence-emitting dye pairs comprise a FRET pair or a quencher/fluor pair. In both cases of a FRET pair and a quencher/fluor pair, the emission spectrum of one of the dyes overlaps a region of the absorption spectrum of the other dye in the pair. As used herein, the term “fluorescence-emitting dye pair” encompasses both a “fluorescence resonance energy transfer (FRET) pair” and a “quencher/fluor pair,” both of which terms are discussed in more detail below. The term “fluorescence-emitting dye pair” is used interchangeably with the phrase “a FRET pair and/or a quencher/fluor pair.”

In some cases (e.g., when the detector ssDNA includes a FRET pair) the labeled detector ssDNA produces an amount of detectable signal prior to being cleaved, and the amount of detectable signal that is measured is reduced when the labeled detector ssDNA is cleaved. In some cases, the labeled detector ssDNA produces a first detectable signal prior to being cleaved (e.g., from a FRET pair) and a second detectable signal when the labeled detector ssDNA is cleaved (e.g., from a quencher/fluor pair). As such, in some cases, the labeled detector ssDNA comprises a FRET pair and a quencher/fluor pair.

In some cases, the labeled detector ssDNA comprises a FRET pair. FRET is a process by which radiationless transfer of energy occurs from an excited state fluorophore to a second chromophore in close proximity. The range over which the energy transfer can take place is limited to approximately 10 nanometers (100 angstroms), and the efficiency of transfer is extremely sensitive to the separation distance between fluorophores. Thus, as used herein, the term “FRET” (“fluorescence resonance energy transfer”; also known as “Förster resonance energy transfer”) refers to a physical phenomenon involving a donor fluorophore and a matching acceptor fluorophore selected so that the emission spectrum of the donor overlaps the excitation spectrum of the acceptor, and further selected so that when donor and acceptor are in close proximity (usually 10 nm or less) to one another, excitation of the donor will cause excitation of and emission from the acceptor, as some of the energy passes from donor to acceptor via a quantum coupling effect. Thus, a FRET signal serves as a proximity gauge of the donor and acceptor; only when they are in close proximity to one another is a signal generated. The FRET donor moiety (e.g., donor fluorophore) and FRET acceptor moiety (e.g., acceptor fluorophore) are collectively referred to herein as a “FRET pair.”

The donor-acceptor pair (a FRET donor moiety and a FRET acceptor moiety) is referred to herein as a “FRET pair” or a “signal FRET pair.” Thus, in some cases, a subject labeled detector ssDNA includes two signal partners (a signal pair), when one signal partner is a FRET donor moiety and the other signal partner is a FRET acceptor moiety. A subject labeled detector ssDNA that includes such a FRET pair (a FRET donor moiety and a FRET acceptor moiety) will thus exhibit a detectable signal (a FRET signal) when the signal partners are in close proximity (e.g., while on the same RNA molecule), but the signal will be reduced (or absent) when the partners are separated (e.g., after cleavage of the RNA molecule by a Type V CRISPR/Cas endonuclease (e.g., a Cas12 protein such as Cas12a, Cas12b, Cas12c, Cas12d, Cas12e)).

FRET donor and acceptor moieties (FRET pairs) will be known to one of ordinary skill in the art and any convenient FRET pair (e.g., any convenient donor and acceptor moiety pair) can be used. Examples of suitable FRET pairs include but are not limited to those presented in Table 1. See also: Bajar et al. Sensors (Basel). 2016 Sep. 14; 16(9); and Abraham et al. PLoS One. 2015 Aug. 3; 10(8):e0134436.

In some cases, a detectable signal is produced when the labeled detector ssDNA is cleaved (e.g., in some cases, the labeled detector ssDNA comprises a quencher/fluor pair). One signal partner of a signal quenching pair produces a detectable signal and the other signal partner is a quencher moiety that quenches the detectable signal of the first signal partner (i.e., the quencher moiety quenches the signal of the signal moiety such that the signal from the signal moiety is reduced (quenched) when the signal partners are in proximity to one another, e.g., when the signal partners of the signal pair are in close proximity).

In some cases, the cleavage of a labeled detector ssDNA can be detected by measuring a colorimetric read-out. For example, the liberation of a fluorophore (e.g., liberation from a FRET pair, liberation from a quencher/fluor pair, and the like) can result in a wavelength shift (and thus color shift) of a detectable signal. Thus, in some cases, cleavage of a subject labeled detector ssDNA can be detected by a color-shift. Such a shift can be expressed as a loss of an amount of signal of one color (wavelength), a gain in the amount of another color, a change in the ratio of one color to another, and the like.

Type V CRISPR/Cas endonucleases are a subtype of Class 2 CRISPR/Cas e endonucleases. For examples of type V CRISPR/Cas systems and the endonucleases (e.g., Cas12 family proteins such as Cas12a), see, e.g., Shmakov et al., Nat Rev Microbiol. 2017 March; 15(3):169-182: “Diversity and evolution of class 2 CRISPR-Cas systems.” Examples include, but are not limited to: Cas12 family (Cas12a, Cas12b, Cas12c), C2c4, C2c8, C2c5, C2c10, and C2c9; as well as CasX (Cas12e) and CasY (Cas12d). Also see, e.g., Koonin et al., Curr Opin Microbiol. 2017 June; 37:67-78: “Diversity, classification and evolution of CRISPR-Cas systems.”

As such in some cases, a subject type V CRISPR/Cas endonucleases is a Cas12 protein (e.g., Cas12a, Cas12b, Cas12c). In some cases, a subject type V CRISPR/Cas endonucleases is a Cas12 protein such as Cas12a, Cas12b, Cas12c, Cas12d, Cas12e, Cas12d, or Cas12e. In some cases, a subject type V CRISPR/Cas endonucleases is a Cas12a protein. In some cases, a subject type V CRISPR/Cas endonucleases is a Cas12b protein. In some cases, a subject type V CRISPR/Cas endonucleases is a Cas12c protein. In some cases, a subject type V CRISPR/Cas endonucleases is a Cas12d protein. In some cases, a subject type V CRISPR/Cas endonucleases is a Cas12e protein. In some cases, a subject type V CRISPR/Cas endonucleases is protein selected from: Cas12 (e.g., Cas12a, Cas12b, Cas12c, Cas12d, Cas12e), C2c4, C2c8, C2c5, C2c10, and C2c9. In some cases, a subject type V CRISPR/Cas endonucleases is protein selected from: C2c4, C2c8, C2c5, C2c10, and C2c9. In some cases, a subject type V CRISPR/Cas endonucleases is protein selected from: C2c4, C2c8, and C2c5. In some cases, a subject type V CRISPR/Cas endonucleases is protein selected from: C2c10 and C2c9.

A Type V CRISPR/Cas endonuclease binds to target DNA at a target sequence defined by the region of complementarity between the DNA-targeting RNA and the target DNA. As is the case for many CRISPR/Cas endonucleases, site-specific binding (and/or cleavage) of a double stranded target DNA occurs at locations determined by both (i) base-pairing complementarity between the guide RNA and the target DNA; and (ii) a short motif [referred to as the protospacer adjacent motif (PAM)] in the target DNA.

In some cases, the PAM for a Type V CRISPR/Cas endonuclease is immediately 5′ of the target sequence (e.g., of the non-complementary strand of the target DNA—the complementary strand hybridizes to the guide sequence of the guide RNA while the non-complementary strand does not directly hybridize with the guide RNA and is the reverse complement of the non-complementary strand). In some cases (e.g., when Cas12a or Cas12b as described herein is used), the PAM sequence is 5′-TTN-3′. In some cases, the PAM sequence is 5′-TTTN-3.′

In some cases, different Type V CRISPR/Cas endonucleases (i.e., Type V CRISPR/Cas endonucleases from various species) may be advantageous to use in the various provided methods in order to capitalize on a desired feature (e.g., specific enzymatic characteristics of different Type V CRISPR/Cas endonucleases). Type V CRISPR/Cas endonucleases from different species may require different PAM sequences in the target DNA. Thus, for a particular Type V CRISPR/Cas endonuclease of choice, the PAM sequence requirement may be different than the 5′-TTN-3′ or 5′-TTTN-3′ sequence described above. Various methods (including in silico and/or wet lab methods) for identification of the appropriate PAM sequence are known in the art and are routine, and any convenient method can be used.

A nucleic acid molecule (e.g., a natural crRNA) that binds to a type V CRISPR/Cas endonuclease (e.g., a Cas12 protein such as Cas12a, Cas12b, Cas12c, Cas12d, Cas12e), forming a ribonucleoprotein complex (RNP), and targets the complex to a specific target sequence within a target DNA is referred to herein as a “guide RNA.” It is to be understood that in some cases, a hybrid DNA/RNA can be made such that a guide RNA includes DNA bases in addition to RNA bases—but the term “guide RNA” is still used herein to encompass such hybrid molecules. A subject guide RNA includes a guide sequence (also referred to as a “spacer”) that hybridizes to target sequence of a target DNA and a constant region (e.g., a region that is adjacent to the guide sequence and binds to the type V CRISPR/Cas endonuclease). A “constant region” can also be referred to herein as a “protein-binding segment.” In some cases, e.g., for Cas12a, the constant region is 5′ of the guide sequence.

The guide sequence has complementarity with (hybridizes to) a target sequence of the target DNA. In some cases, the guide sequence is 15-28 nucleotides (nt) in length (e.g., 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 nt in length). In some cases, the guide sequence is 18-24 nucleotides (nt) in length. In some cases, the guide sequence is at least 15 nt long (e.g., at least 16, 18, 20, or 22 nt long). In some cases, the guide sequence is at least 17 nt long. In some cases, the guide sequence is at least 18 nt long. In some cases, the guide sequence is at least 20 nt long.

In some cases, the guide sequence has 80% or more (e.g., 85% or more, 90% or more, 95% or more, or 100% complementarity) with the target sequence of the target DNA. In some cases, the guide sequence is 100% complementary to the target sequence of the target DNA. In some cases, the target DNA includes at least 15 nucleotides (nt) of complementarity with the guide sequence of the guide RNA.

CRISPR-diagnostic assays have gained significant interest in the last few years. This interest has grown rapidly during the current COVID-19 pandemic, where CRISPR-diagnostics have been frontline contenders for rapid testing solutions. This surge in CRISPR-diagnostic research prompts what are the achievable limits of detection and associated assay times achievable by the kinetics of CRISPR endonucleases, such as Cas12 and Cas13. To that end, the disclosure describes a model based on Michaelis-Menten enzyme kinetics theory applied to CRISPR endonucleases. This model was used to develop analytical solutions for reaction kinetics and develop simplified calculations to validate consistency in reported enzyme kinetic parameters.

The analysis disclosed herein was applied to reported studies of Michaelis-Menten-type kinetic data for CRISPR endonucleases, particularly, Cas. These studies include all subtypes CRISPR endonucleases, such as Cas12, Cas13, and orthologs. All but one study clearly violated at least two of our three rules and therefore present data that violate basic physical limits.

A study of reaction kinetics of LbCas12a was performed with both ssDNA and dsDNA activators. These data was used to validate the model and its predicted scaling. The validated model was used to explore CRISPR reaction time scales and the degree of reaction completion for practically relevant target concentrations applicable to CRISPR-diagnostic assays. The results have broad implications for achievable limits of detection and assay times of emerging, amplification-free CRISPR-detection methods.

As applications for CRIPSR-diagnostics are explored, SNPs detection is of interest. The specificity of Cas enzymes is well suited to target these mutations which are implicated in, for example, SARS-CoV-2 variants and some cancers. CRISPR-based SNP detection assays, however, often rely on endpoint measurements to differentiate WT and SNP targets. As disclosed herein, this detection scheme is hindered by irregular experimental parameters (such as assay initiation and duration as well as target concentrations) which can be difficult to choose or control.

To address these limitations, the disclosure provides an assay for the detection of SNPs based on fundamental Michalis-Menten enzyme kinetics.

In the Examples described below, such assays are demonstrated to differentiate a 20-base pair WT target from all 60 of its associated mutants. Specifically, endpoint-based assays failed to identify SNP targets; however, kinetic parameters are affected by WT versus SNP activation. Therefore, these kinetic parameters provide a greater dynamic range for SNP detection. For example, the K_{M} measured for WT is 23-fold greater than the highest K_{M} measured among all SNPs. This compares to only 7.8-fold difference on endpoint fluorescence.

Similarly, k*_{cat}/K_{M} results in a 130-fold differences between WT and the lowest k*_{cat}/K_{M} measured among all SNPs. Therefore, the assays disclosed herein provide a time- and concentration-insensitive measurement ideal to differentiate WT from SNPs.

Accordingly, certain aspects of the disclosure provide a method of analyzing a target nucleic acid, the method comprising, using the target nucleic acid as a reagent and a reporter as a substrate, assaying an enzyme kinetic parameter of a CRISPR endonuclease comprising a guide RNA (gRNA) that hybridizes with a reference nucleic acid.

In some cases, an enzyme kinetic parameter is the rates of cleavage of the reporter at different concentrations of the reporter. An enzyme kinetic parameter can also be the initial rates of cleavage of the reporter at different concentrations of the reporter, such as, rates of cleavage of the reporter during initial 1 to 10 minutes, such as initial 1, 2, 3, 4, 5, 6, 7, 8, 9, or 10 minutes after initiation of the CRISPR endonuclease mediated cleavage of the reporter.

In some cases, an enzyme kinetic parameter is a quantitative measure of the rates of cleavage of the reporter at different concentrations of the reporter. An enzyme kinetic parameter can also be the initial rates of cleavage of the reporter at different concentrations of the reporter, such as, rates of cleavage of the reporter during initial 1 to 10 minutes, such as initial 1, 2, 3, 4, 5, 6, 7, 8, 9, or 10 minutes after initiation of the CRISPR endonuclease mediated cleavage of the reporter.

In some cases, the quantitative measure is a goodness of fit parameter for the enzyme activity. For example, a goodness of fit parameter can indicate how accurately the data of initial velocities at a plurality of reporter concentrations fits the Michaelis Menten model. For example, the goodness of fit parameter can be MSE or X2. MSE describes the Mean Squared Error that is a statistical measure of the amount of error between the model and the data. λ2 describes the Chi-square statistical method assessing the goodness of fit between observed values and those expected theoretically.

In some cases, the enzyme kinetic parameter is the Michaelis-Menten constant (K_{M}) of the CRISPR endonuclease.

In certain cases, the enzyme kinetic parameter is the apparent turnover rate (k*_{cat}) of the CRISPR endonuclease.

In further cases, the enzyme kinetic parameter further comprises apparent catalytic efficiency (k*_{cat}/K_{M}).

The enzyme kinetic parameter can also be turnover rate (k_{cat}) or catalytic efficiency (k_{cat}/K_{M}).

A target nucleic acid can be any nucleic acid of interest, i.e., a nucleic acid to be analyzed. In certain cases, a target nucleic acid comprises a stretch of nucleotides that is targeted using a gRNA in a CRISPR endonuclease. Typically, a target sequence is a stretch of 15 to 25 nucleotides in the target nucleic acid. A gRNA specifically hybridizes to the target sequence, the details of which are described above.

Non-limiting examples of target nucleic acids include a genome, such as genomic DNA or RNA, wherein the target sequence targeted by a gRNA is a sequence of interest in the genome. The genomic nucleic acid can be eukaryotic genome, bacterial genome, viral genome, or fungal genome. Additional genomic nucleic acids are known in the art and analyzing such nucleic acids using the methods described herein is within the purview of the disclosure.

In some cases, the target nucleic acid comprises a target sequence that is known to be mutated in the analyzed genome. For example, a target sequence may include nucleotide positions that are mutated in a virus to produce variant viruses. Alternatively, a target sequence may include nucleotide positions that exhibit single nucleotide polymorphism.

In some cases, the target nucleic acid comprises a tumor marker, i.e., a mutation in the genome that indicates increased likelihood that a tumor develops in a subject. Various tumor markers are well known in the art, for example, BRCA mutation, which indicates increased likelihood of the development of breast or ovarian cancer and EGFR mutation, which indicates increased likelihood of the development of certain kind of lung cancers.

In some cases, the target nucleic acid is amplified using an enzymatic exponential nucleic acid amplification before using the target nucleic acid amplicon as the reagent in assaying the enzyme kinetic parameter of the CRISPR endonuclease. Such amplified target nucleic acid can then be used in assaying the enzyme kinetic parameter of the CRISPR endonuclease.

In some cases, based on the assayed kinetic parameter of the CRISPR endonuclease, the target nucleic acid is identified as a wild-type nucleic acid. In other cases, based on the assayed kinetic parameter of the CRISPR endonuclease, the target nucleic acid is identified as a mutant or variant nucleic acid. For example, the enzyme kinetic parameter of the target nucleic acid can be compared to the known or expected kinetic parameter of the reference nucleic acid.

In certain embodiments, the gRNA is designed to provide 100% sequence complementarity to a reference nucleic acid. The reference nucleic acid can be a WT nucleic acid or a specific variant nucleic acid. Generally, a CRISPR endonuclease has the best kinetic properties, i.e., the lowest K_{M}, the highest k*_{cat}, the highest k*cₐₜ/K_{M}, the highest k_{cat}, and/or the highest k_{cat}/K_{M} for the reference nucleic acid. Therefore, if a target nucleic acid exhibits the best kinetic properties, i.e., the lowest K_{M}, the highest k*_{cat}, the highest k*_{cat}/K_{M}, the highest k_{cat}, and/or the highest k_{cat}/K_{M}, then the target nucleic acid is identified as having the same sequence as the reference nucleic acid. If a target nucleic acid does not exhibit the best kinetic properties, i.e., the lowest K_{M}, the highest k*_{cat}, the highest k*_{cat}/K_{M}, the highest k_{cat}, and/or the highest k_{cat}/K_{M}, then the target nucleic acid is identified as having a different sequence than the reference nucleic acid.

In some cases, enzyme kinetic parameters of a CRISPR endonuclease for a number of variants are pre-determined. Then the enzyme kinetic parameter for a tested target nucleic acid can be compared with the pre-determined kinetic parameters. Based on the matching kinetic parameters, the tested target nucleic acid can be identified as the nucleic acid for which the CRISPR endonuclease exhibits the assayed kinetic parameters.

In some embodiments, the concentration of the target nucleic acid is not known or cannot be estimated. In some embodiments, the concentration of the target nucleic acid is known or can be estimated.

In certain such embodiments, assaying the enzyme kinetic parameter of the CRISPR endonuclease comprises assaying the CRISPR endonuclease with different dilutions of the reporter. The different dilutions of the reporter can be used to determine one or more kinetic parameters of the CRISPR endonuclease.

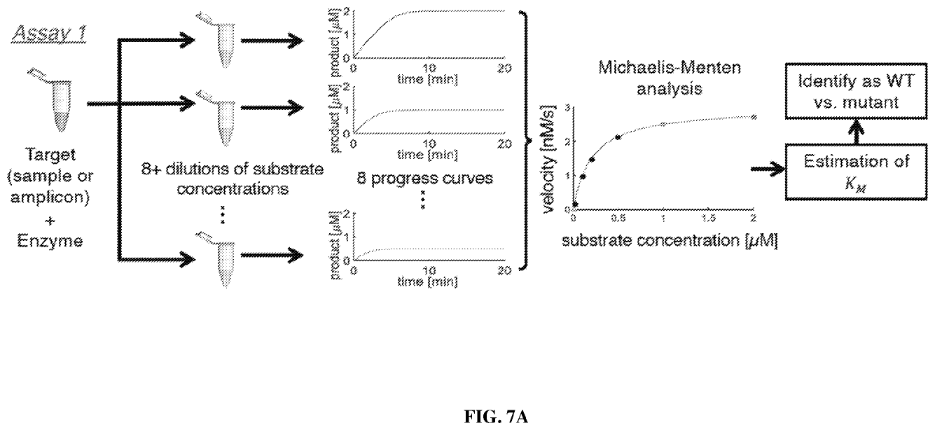

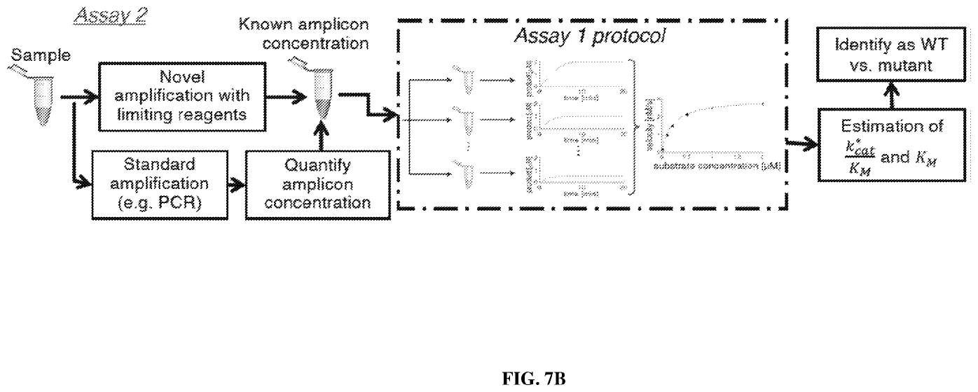

In some embodiments as exemplified in FIG. 7A, the concentration of the target nucleic acid is not known or cannot be estimated. In certain such embodiments, assaying the enzyme kinetic parameter of the CRISPR endonuclease comprises assaying the CRISPR endonuclease with different dilutions of the reporter. Such assays can be used to measure one or more kinetic parameters of the CRISPR endonuclease.

In some embodiments as exemplified in FIG. 7B, the concentration of target nucleic acid can be estimated, for example, by amplifying the nucleic acid in a sample using an enzymatic exponential nucleic acid amplification and then quantifying the nucleic acid. Different known amplificon concentrations can then be used in varying concentrations of the reporters to obtain enzyme kinetics of a CRISPR endonuclease and to calculate one or more enzyme kinetic parameters of the CRISPR endonuclease.

Any suitable CRISPR endonuclease can be used in the methods disclosed herein. For example, the CRISPR endonuclease can be a type V CRISPR/Cas endonuclease, such as a Cas12, Cas12a (Cpf1), Cas12b (C2c1), a Cas12d (CasY), or Cas12e (CasX).

Any suitable reporter as discussed herein could be used for measuring CRISPR endonuclease activity. For example, a reporter can produce a detectable signal after being cleaved. In some cases, the reporter comprises a fluorescence-emitting dye pair, such as a FRET pair. The FRET pair can be a quencher/fluor pair.

Further aspects of the disclosure provide a method of a comparing a sequence of a target nucleic acid with a sequence of a reference nucleic acid, comprising contacting the target nucleic acid as a reagent and a plurality of concentrations of a reporter as a substrate with a Clustered Regularly Interspaced Short Palindromic Repeats (CRISPR) endonuclease comprising a guide RNA (gRNA) that hybridizes with the reference nucleic acid, and quantifying the rates of cleavage of the reporter at the plurality of concentrations of the reporter.

In some cases, the method comprises quantifying the initial rates of cleavage of the reporter at a plurality of the reporter concentrations. Initial rates of cleavage includes the rates of cleavage within the first 1 to 10 minutes, such as 1, 2, 3, 4, 5, 6, 7, 8, 9, or 10 minutes after initiation of the CRISPR endonuclease mediated cleavage of the reporter.

In certain such method, a difference in the rates of cleavage by the CRISPR endonuclease of the reporter at the plurality of concentrations of the reporter in the presence of the target nucleic acid as compared to the rates of cleavage of the reporter by the CRISPR endonuclease at the plurality of concentrations of the reporter in the presence of the reference nucleic acid indicates that the sequence of the target nucleic acid is different from the sequence of the reference nucleic acid.

In certain such method, no difference in the rates of cleavage by the CRISPR endonuclease of the reporter at the plurality of concentrations of the reporter in the presence of the target nucleic acid as compared to the rates of cleavage of the reporter by the CRISPR endonuclease at the plurality of concentrations of the reporter in the presence of the reference nucleic acid indicates that the sequence of the target nucleic acid is the same as the sequence of the reference nucleic acid.

In some cases, the plurality of concentrations of the reporter comprises a reporter concentration higher than the K_{M} of the CRISPR endonuclease and at least one reporter concentration is approximately equal to or smaller than the K_{M} of the CRISPR endonuclease.

Details of the methods discussed above for analyzing a target nucleic acid are also applicable to the methods of comparing a sequence of a target nucleic acid with a sequence of a reference nucleic acid. Embodiments using such details in the methods of comparing the sequences are also within the purview of the disclosure.

In certain cases, the methods disclosed herein comprise denaturation of a double stranded target DNA, for example, genomic DNA isolated from human cells, into single stranded DNA before kinetic properties of a CRISPR endonuclease are measured. Many such denaturation agents are known in the art and inclusion of such agents in the methods disclosed herein is within the purview of the invention.

Further aspects of the disclosure provide a kit suitable for performing the methods disclosed herein. Certain such kits comprise:
a reporter;
a CRISPR endonuclease comprising a guide RNA comprising a sequence that is perfectly complementary to a reference nucleic acid; and
a non-transitory computer readable media comprising instructions, which when executed by a processor, cause the processor to analyze the results of the assay of the CRISPR endonuclease using a target nucleic acid as a reagent and the reporter molecule as a substrate and provide an enzyme kinetic parameter of the CRISPR endonuclease.

The aspects described above with respect to methods disclosed herein are also applicable to the kits disclosed herein and such embodiments are within the purview of the disclosure.

In some cases, the kit comprises different concentrations of the reporters that can be readily mixed with appropriate reactions. In certain embodiments, kits can also contain various buffers that can be used to dilute or treat samples. Kits may also contain reagents for amplifying a target nucleic acid, for example, using a polymerase chain reaction.

In certain cases, the kits comprise a denaturation agent that can be used for denaturing a double stranded target DNA, for example, genomic DNA isolated from human cells, into single stranded DNA. Many such denaturation agents are known in the art and inclusion of such agents in the kits disclosed herein is within the purview of the invention.

The non-transitory computer readable media comprising instructions that cause a processor to perform the methods of the invention can be provided in the form of “programming,” where the term “computer-readable media” as used herein refers to any non-transitory storage or transmission media that participates in providing instructions and/or data to a computer for execution and/or processing. Examples of storage media include a hard disk, optical disk, magneto-optical disk, CD-ROM, CD-R, magnetic tape, non-volatile memory card, ROM, DVD-ROM, Blue-ray disk, solid state disk, network attached storage (NAS), etc., whether such devices are internal or external to the computer. A file containing information can be “stored” on computer readable medium, where “storing” means recording information such that it is later accessible and retrievable by a computer.

The instructions may be in the form of programming that is written in one or more of any number of computer programming languages. Such languages include, for example, Java (Sun Microsystems, Inc., Santa Clara, CA), Visual Basic (Microsoft Corp., Redmond, WA), and C++(AT&T Corp., Bedminster, NJ), as well as many others.

The present disclosure also provides computer devices. The computer devices include one or more processors and any of the non-transitory computer readable media of the present disclosure. Accordingly, in some embodiments, the computer devices can perform any of the methods described in the Methods section herein.

In certain aspects, a computer device of the present disclosure is a local computer device, preferably, a portable computer device, such as a smart-phone or table. In some embodiments, the computer device is a remote computer device (e.g., a remote server), meaning that the instructions are executed on a computer device different from a local computer device and/or the instructions are downloadable from the remote computer device to a local computer device, e.g., for execution on the local computer device. In some embodiments, the instructions constitute a web-based application stored on a remote server.

Notwithstanding the appended claims, the disclosure is also defined by the following Embodiments:

Embodiment 1. A method of analyzing a target nucleic acid, the method comprising, using the target nucleic acid as a reagent and a reporter as a substrate, assaying an enzyme kinetic parameter of a Clustered Regularly Interspaced Short Palindromic Repeats (CRISPR) endonuclease comprising a guide RNA (gRNA) that hybridizes with a reference nucleic acid.

Embodiment 2. A method of comparing a sequence of a target nucleic acid with a sequence of a reference nucleic acid, comprising contacting the target nucleic acid as a reagent and a plurality of concentrations of a reporter as a substrate with a Clustered Regularly Interspaced Short Palindromic Repeats (CRISPR) endonuclease comprising a guide RNA (gRNA) that hybridizes with the reference nucleic acid, and quantifying an enzyme kinetic parameter of the CRISPR endonuclease.

Embodiment 3. The method of Embodiment 1 or 2, wherein the enzyme kinetic parameter comprises the rates of cleavage of the reporter at different concentrations of the reporter.

Embodiment 4. The method of any one of the preceding Embodiments, wherein the enzyme kinetic parameter comprises the initial rates of cleavage of the reporter at different concentrations of the reporter.

Embodiment 5. The method of Embodiment 4, wherein the initial rates of cleavage of the reporter comprise the rates of cleavage of the reporter during the initial 1 to 10 minutes after initiation of the CRISPR endonuclease mediated cleavage of the reporter.

Embodiment 6. The method of any one of the preceding Embodiments, wherein the enzyme kinetic parameter is the Michaelis-Menten constant (K_{M}) of the CRISPR endonuclease.

Embodiment 7. The method of any one of the preceding Embodiments, wherein the enzyme kinetic parameter is the apparent enzyme turnover rate (k*_{cat}) of the CRISPR endonuclease.

Embodiment 8. The method of any one of the preceding Embodiments, wherein the enzyme kinetic parameter further comprises apparent catalytic efficiency (k*_{cat}/K_{M}).

Embodiment 9. The method of any one of the preceding Embodiments, wherein the enzyme kinetic parameter is the enzyme turnover rate (k_{cat}) of the CRISPR endonuclease.

Embodiment 10. The method of any one of the preceding Embodiments, wherein the enzyme kinetic parameter further comprises catalytic efficiency (k_{ca}t/K_{M}).

Embodiment 11. The method of any one of the preceding Embodiments, wherein the target nucleic acid is amplified using an enzymatic exponential nucleic acid amplification before using the target nucleic acid amplicon as the reagent in assaying the enzyme kinetic parameter of the CRISPR endonuclease.

Embodiment 12. The method of any one of the preceding Embodiments, wherein analyzing the target nucleic acid comprises identifying the target nucleic acid as having the same sequence as the reference nucleic acid.

Embodiment 13. The method of any one of Embodiments 1 to 11, wherein analyzing the target nucleic acid comprises identifying the target nucleic acid as having a different sequence from the reference nucleic acid.

Embodiment 14. The method of any one of the preceding Embodiments, wherein assaying the enzyme kinetic parameter of the CRISPR endonuclease comprises assaying enzyme activity of the CRISPR endonuclease using the same concentration of the target nucleic acid at different concentrations of the reporter.

Embodiment 15. The method of any one of the preceding Embodiments, wherein assaying the enzyme kinetic parameter of the CRISPR endonuclease comprises assaying enzyme activity of the CRISPR endonuclease under conditions of excess amount of the target nucleic acid and/or excess amount of the reporter as compared to the amount of the CRISPR endonuclease.

Embodiment 16. A method of comparing a sequence of a target nucleic acid with a sequence of a reference nucleic acid, comprising contacting the target nucleic acid as a reagent and a plurality of concentrations of a reporter as a substrate with a Clustered Regularly Interspaced Short Palindromic Repeats (CRISPR) endonuclease comprising a guide RNA (gRNA) that hybridizes with the reference nucleic acid, and quantifying the rates of cleavage of the reporter at the plurality of concentrations of the reporter.

Embodiment 17. The method of Embodiment 16, comprising quantifying the initial rates of cleavage of the reporter at the plurality of concentrations of the reporter.

Embodiment 18. The method of Embodiment 16 or 17, wherein the initial rates of cleavage of the reporter comprise the rates of cleavage of the reporter during the initial 1 to 10 minutes after initiation of the CRISPR endonuclease mediated cleavage of the reporter.

Embodiment 19. The method of any one of Embodiments 16 to 18, wherein a difference in the rates of cleavage by the CRISPR endonuclease of the reporter at the plurality of concentrations of the reporter in the presence of the target nucleic acid as compared to the rates of cleavage of the reporter by the CRISPR endonuclease at the plurality of concentrations of the reporter in the presence of the reference nucleic acid indicates that the sequence of the target nucleic acid is different from the sequence of the reference nucleic acid.

Embodiment 20. The method of any one of Embodiments 16 to 18, wherein no difference in the rates of cleavage by the CRISPR endonuclease of the reporter at the plurality of concentrations of the reporter in the presence of the target nucleic acid as compared to the rates of cleavage of the reporter by the CRISPR endonuclease at the plurality of concentrations of the reporter in the presence of the reference nucleic acid indicates that the sequence of the target nucleic acid is the same as the sequence of the reference nucleic acid.

Embodiment 21. The method of any one of the Embodiments 16 to 20, wherein the plurality of concentrations of the reporter comprises a reporter concentration higher than the K_{M} of the CRISPR endonuclease and at least one reporter concentration is approximately equal to or smaller than the K_{M} of the CRISPR endonuclease.

Embodiment 22. The method of any one of the preceding Embodiments, wherein the CRISPR endonuclease is a type V CRISPR/Cas endonuclease.

Embodiment 23. The method of Embodiment 22, wherein the type V CRISPR/Cas endonuclease is a Cas12.

Embodiment 24. The method of Embodiment 22, wherein the type V CRISPR/Cas endonuclease is a Cas12a (Cpf1) or Cas12b (C2c1) protein.

Embodiment 25. The method of Embodiment 22, wherein the type V CRISPR/Cas endonuclease is a Cas12d (CasY) or Cas12e (CasX) protein.

Embodiment 26. The method of any one of the preceding Embodiments, wherein the reporter produces a detectable signal after being cleaved.

Embodiment 27. The method of Embodiment 26, wherein the reporter comprises a fluorescence-emitting dye pair Embodiment 28. The method of Embodiment 27, wherein the fluorescence-emitting dye pair is a fluorescence resonance energy transfer (FRET) pair.

Embodiment 29. The method of Embodiment 28, wherein the FRET pair is a quencher/fluor pair.

Embodiment 30. The method of any one of the preceding Embodiments, wherein the target nucleic acid is denatured from dsDNA to ssDNA before using the denatured target nucleic acid as a reagent in assaying the enzyme kinetic parameter of the CRISPR endonuclease.

Embodiment 31. A kit comprising:
a reporter;
a CRISPR endonuclease comprising a guide RNA comprising a sequence that is perfectly complementary to a reference nucleic acid; and
a non-transitory computer readable media comprising instructions, which when executed by a processor, cause the processor to analyze the results of the assay of the CRISPR endonuclease using a target nucleic acid and the reporter molecule as a substrate and provide an enzyme kinetic parameter of the CRISPR endonuclease.

Embodiment 32. The kit of Embodiment 31, wherein the enzyme kinetic parameter is the Michaelis-Menten constant (K_{M}) of the CRISPR endonuclease.

Embodiment 33. The kit of Embodiment 31 or 32, wherein the enzyme kinetic parameter is the apparent enzyme turnover rate (k*_{cat}) of the CRISPR endonuclease.

Embodiment 34. The kit of any one of Embodiments 31 to 33, wherein the enzyme kinetic parameter further comprises apparent catalytic efficiency (k*ca/K_{M}).

Embodiment 35. The kit of Embodiment 31 or 34, wherein the enzyme kinetic parameter is the enzyme turnover rate (k_{cat}) of the CRISPR endonuclease.

Embodiment 36. The kit of Embodiment 31 or 35, wherein the enzyme kinetic parameter further comprises catalytic efficiency (k_{cat}/K_{V}).

Embodiment 37. The kit of any one of Embodiments 31 to 36, wherein the enzyme kinetic parameter comprises the rates of cleavage of the reporter at different concentrations of the reporter.

Embodiment 38. The kit of any one of Embodiments 31 to 37, wherein the enzyme kinetic parameter comprises the initial rates of cleavage of the reporter at different concentrations of the reporter.

Embodiment 39. The kit of Embodiment 38, wherein the initial rates of cleavage of the reporter comprise the rates of cleavage of the reporter during the initial 1 to 10 minutes after initiation of the CRISPR endonuclease mediated cleavage of the reporter.

Embodiment 40. The kit of any one of Embodiments 31 to 39, wherein the non-transitory computer readable media comprises instructions, which when executed by a processor, cause the processor analyzing the target nucleic acid comprises identifying the target nucleic acid as having the same sequence as the reference nucleic acid.

Embodiment 41. The kit of any one of Embodiments 31 to 39, wherein the non-transitory computer readable media comprises instructions, which when executed by a processor, cause the processor analyzing the target nucleic acid comprises identifying the target nucleic acid as having a different sequence from the reference nucleic acid.

Embodiment 42. The kit of any one of Embodiments 31 to 41, wherein the CRISPR endonuclease is a type V CRISPR/Cas endonuclease.

Embodiment 43. The kit of Embodiment 42, wherein the type V CRISPR/Cas endonuclease is a Cas12.

Embodiment 44. The kit of Embodiment 42, wherein the type V CRISPR/Cas endonuclease is a Cas12a (Cpf1) or Cas12b (C2c1) protein.

Embodiment 45. The kit of Embodiment 42, wherein the type V CRISPR/Cas endonuclease is a Cas12d (CasY) or Cas12e (CasX) protein.

Embodiment 46. The kit of any one of the Embodiments 31 to 45, wherein the reporter molecule produces a detectable signal after being cleaved.

Embodiment 47. The kit of Embodiment 46, wherein the reporter molecule comprises a fluorescence-emitting dye pair.

Embodiment 48. The kit of Embodiment 47, wherein the fluorescence-emitting dye pair is a fluorescence resonance energy transfer (FRET) pair.

Embodiment 49. The kit of Embodiment 48, wherein the FRET pair is a quencher/fluor pair.

Embodiment 50. The kit of any one of Embodiments 31 to 49, further comprising a denaturing agent to transform extracted target DNA from a double stranded form to a single stranded form.

### EXAMPLES

The following examples are put forth to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### Example 1—CRISPR-Based Diagnostics for COVID-19

The global spread of COVID-19 highlighted the need for early-stage, rapid, and specific testing assays. CRISPR-based diagnostic techniques stand out among other nucleic acid detection methods for their high specificity and their compatibility with point-of-care devices. Despite limited sensitivity without pre-amplification, CRISPR-based diagnostics has recently been established as a leading candidate for the detection of SARS-CoV-2 and its variants. Its high specificity also makes CRISPR one of the main contenders for the detection of SNPs. The capability to differentiate small mutations may be critical to the identification of new virus variants. For example, recent SARS-CoV-2 variants differ from the original sequence by just a few nucleotides. Aside from infectious disease assays, the ability to differentiate SNPs also plays an important role in cancer detection and genotyping. Several groups reported CRISPR based detection of SNPs. In particular, Cas12a, Cas13a, and Cas14a have been used to confirm that CRISPR-based assays are sufficiently specific for SNP detection. However, to date, the differentiation among mutants has been limited to the use of endpoint fluorescent signal assays which quantify the sum of enzyme activity over some specified time. The detailed effects of target mutations on Cas enzyme trans-cleavage kinetic rates has not been elucidated. No systematic study was performed for detecting single base-pair mutations based on the Michaelis-Menten kinetics of CRISPR enzymes.

This Example systematically describes the effects of SNPs on LbCas12a enzyme trans-cleavage activity. Specifically, a gRNA comprising a 20-base pair DNA sequence was used to target WT as well as all 60 related SNPs for that sequence. CRISPR assay conditions where endpoint detection of WT and SNP targets appears indistinguishable were first identified. Then, the use of a Michaelis-Menten model to measure the K_{M} and apparent catalytic efficiency k*_{cat}/K_{M} of WT- and SNP-activated Cas12a enzymes. Importantly, K_{M} and k at/K_{M} discriminated WT from mutants in a manner superior to differentiation based on endpoint detection. K_{M} is by itself a discriminating criterion which does not require knowledge of target concentrations. k*_{cat}/K_{M} however, offers the clearest differentiation between the WT and mutants. Overall, this Example demonstrates SNP effects on the affinity and catalytic efficiencies of trans-cleavage and demonstrates a new modality for CRISPR diagnostics.

### Materials and Methods

### Complexing and Activating Cas12-gRNA

LbCas12a was purchased from New England Biolabs (MA, USA) at a concentration of 100 μM. RNA oligonucleotides were purchased from GeneLink (FL, USA) and resuspended to 100 μM in RNA reconstitution buffer (GeneLink). ssDNA WT and SNP targets were purchased from Elim Biopharmaceuticals Inc. (CA, USA) at 100 μM. A complete list of oligos is provided in Table 1.

TABLE 1
Confidence intervals for the measured kinetic parameters of
the WT and all SNPs. Confidence intervals are estimates obtained
from GraphPad Prism 9 (GraphPad Software, CA, USA) using the
asymmetrical (profile-likelihood) confidence interval option. Summary of
experimentally measured kinetic parameters. This table reports the kinetic
enzyme parameters and 95% confidence intervals on k*_{cat} and
K_{M} measurements.

| | Best fit | | 95% confidence intervals | |
|---|---|---|---|---|
| Target | K*_{cat} (s⁻¹) | K_{M} (nM) | K*_{cat} (s⁻¹ ) | K_{M} (nM) |
| WT | 0.046 | 53 | 0.042 to 0.049 | 39 to 72 |
| G1A | 0.067 | 620 | 0.05 to 0.098 | 310 to 1300 |
| G1T | 0.05 | 430 | 0.045 to 0.055 | 340 to 540 |
| G1C | 0.08 | 540 | 0.071 to 0.091 | 410 to 730 |
| T2A | 0.012 | 140 | 0.01 to 0.013 | 93 to 200 |
| T2G | 0.019 | 130 | 0.018 to 0.021 | 95 to 180 |
| T2C | 0.019 | 100 | 0.018 to 0.02 | 80 to 130 |
| A3T | 0.022 | 340 | 0.02 to 0.025 | 250 to 460 |
| A3G | 0.036 | 220 | 0.034 to 0.038 | 180 to 270 |
| A3C | 0.02 | 210 | 0.019 to 0.022 | 160 to 270 |
| A4T | 0.013 | 230 | 0.012 to 0.015 | 160 to 330 |
| A4G | 0.065 | 1100 | 0.051 to 0.092 | 640 to 1900 |
| A4C | 0.038 | 720 | 0.033 to 0.045 | 510 to 1000 |
| C5A | 0.009 | 260 | 0.008 to 0.01 | 190 to 340 |
| C5T | 0.01 | 480 | 0.009 to 0.011 | 390 to 580 |
| C5G | 0.018 | 100 | 0.017 to 0.02 | 73 to 140 |
| T6A | 0.013 | 360 | 0.011 to 0.014 | 270 to 480 |
| T6G | 0.013 | 530 | 0.012 to 0.015 | 410 to 700 |
| T6C | 0.028 | 150 | 0.026 to 0.03 | 120 to 200 |
| A7T | 0.013 | 180 | 0.012 to 0.014 | 140 to 220 |
| A7G | 0.036 | 71 | 0.033 to 0.039 | 49 to 100 |
| A7C | 0.025 | 110 | 0.023 to 0.028 | 74 to 150 |
| G8A | 0.006 | 200 | 0.004 to 0.01 | 51 to 710 |
| G8T | 0.009 | 89 | 0.008 to 0.01 | 55 to 140 |
| G8C | 0.004 | 130 | 0.003 to 0.005 | 46 to 320 |
| C9A | 0.015 | 470 | 0.013 to 0.018 | 320 to 690 |
| C9T | 0.17 | 570 | 0.14 to 0.21 | 320 to 990 |
| C9G | 0.23 | 1200 | 0.19 to 0.28 | 840 to 1800 |
| A10T | 0.007 | 310 | 0.007 to 0.008 | 220 to 430 |
| A10G | 0.014 | 300 | 0.013 to 0.015 | 250 to 350 |
| A10C | 0.019 | 140 | 0.017 to 0.021 | 96 to 210 |
| A11T | 0.015 | 520 | 0.013 to 0.019 | 330 to 860 |
| A11G | 0.021 | 510 | 0.018 to 0.023 | 380 to 700 |
| A11C | 0.029 | 420 | 0.026 to 0.031 | 340 to 530 |
| G12A | 0.009 | 410 | 0.008 to 0.01 | 290 to 600 |
| G12T | 0.015 | 410 | 0.013 to 0.017 | 320 to 550 |
| G12C | 0.004 | 550 | 0.003 to 0.005 | 330 to 950 |
| A13T | 0.009 | 220 | 0.008 to 0.01 | 160 to 310 |
| A13G | 0.014 | 340 | 0.012 to 0.016 | 230 to 490 |
| A13C | 0.022 | 110 | 0.02 to 0.024 | 74 to 150 |
| A14T | 0.025 | 540 | 0.02 to 0.032 | 330 to 910 |
| A14G | 0.049 | 220 | 0.045 to 0.054 | 170 to 280 |
| A14C | 0.057 | 140 | 0.053 to 0.062 | 107 to 180 |
| T15A | 0.016 | 200 | 0.013 to 0.019 | 130 to 310 |
| T15G | 0.01 | 180 | 0.009 to 0.013 | 120 to 290 |
| T15C | 0.037 | 63 | 0.034 to 0.041 | 44 to 88 |
| A16T | 0.015 | 170 | 0.014 to 0.017 | 120 to 240 |
| A16G | 0.031 | 70 | 0.028 to 0.034 | 47 to 103 |
| A16C | 0.027 | 88 | 0.024 to 0.03 | 61 to 120 |
| C17A | 0.023 | 43 | 0.02 to 0.026 | 25 to 72 |
| C17T | 0.033 | 120 | 0.03 to 0.035 | 94 to 160 |
| C17G | 0.038 | 54 | 0.035 to 0.04 | 42 to 70 |
| C18A | 0.016 | 89 | 0.015 to 0.017 | 70 to 110 |
| C18T | 0.025 | 75 | 0.023 to 0.026 | 60 to 94 |
| C18G | 0.025 | 60 | 0.023 to 0.026 | 47 to 77 |
| A19T | 0.043 | 150 | 0.037 to 0.049 | 99 to 240 |
| A19G | 0.067 | 210 | 0.063 to 0.073 | 170 to 260 |
| A19C | 0.068 | 150 | 0.062 to 0.075 | 110 to 210 |
| C20A | 0.029 | 63 | 0.02 to 0.039 | 15 to 210 |
| C20T | 0.041 | 120 | 0.039 to 0.044 | 94 to 140 |
| C20G | 0.039 | 77 | 0.036 to 0.041 | 61 to 98 |

Complexing and Activating Protocol were as Follows.

100 nM solutions of the Cas12-gRNA complex was prepared by incubating a mixture of 1 μM Cas12 with at least 5-fold excess of synthetic gRNA at 37° C. for 30 min on a hot plate. Cas12-gRNA complexes were then activated for trans-cleavage activity after incubation with either WT target or one of the 61 SNPs. Specifically, Cas12-gRNA complexes were incubated with at least 5-fold excess of the synthetic ssDNA at 37° C. for 30 min on a hot plate. The latter step yielded a solution with an activated Cas12 concentration of 50 nM.

### Trans-Cleavage Kinetics Experiments

The trans-cleavage kinetics assay were performed using 2 nM of activated Cas12 and varied ssDNA reporter concentrations of 13, 25, 50, 100, 200, 400, 800, and 1600 nM. Three replicates were taken for each concentration. The final volume of each reaction was 30 μL. All reactions were buffered in 1× NEBuffer 2.1 (composed of 50 mM NaCl, 10 mM Tris-HCl, 10 mM MgCl2, and 100 g/mL bovine serum albumin at pH=7.9). All trans-cleavage reactions were run at 37° C. Cas12 kinetics data was measured using a 7500 Fast Real-Time PCR system (Applied Biosystems, CA, USA), and fluorescence was measured every 60 s.

### Analysis of Progress Curves

All trans-cleavage and calibration data points were first background and flatfield corrected. Thermal cycler showed significant well-to-well (position-to-position) differences in fluorescence signals for equal concentrations of fluorophores. Hence, flatfield correction refers to a correction for (repeatable and measurable) non-uniform response of the instrument across wells. The discrete array of fluorescence values was considered an “image” of the wells. To obtain a background image, wells were filled with 1× NEBuffer 2.1. For the flat field image, wells were filled with 1.6 μM fluorescein. The correction is then introduced as follows:

$$I = \frac{R - BG}{FF - BG} , \qquad ( 2 )$$

In Equation 2, 1 is the corrected image, R is the raw image, BG is the background image, and FF is the flatfield image. Note that in Equation 3 below, only I and R contain time series data (since BG and FF were averaged in time).

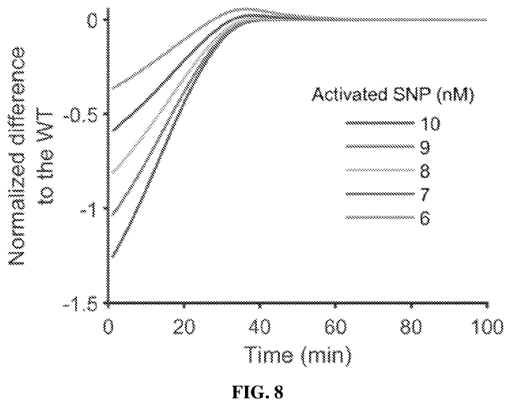

A fluorescence calibration curve was then used to convert fluorescence values (in AU) to molar concentrations. To this end, two nonlinear fits were performed on calibration data, one each for cleaved and uncleaved reporters (FIG. 8). The power law fit has the following form:

$${F}_{i} = \frac{{m}_{i} {c}_{i}}{{10}^{{c}_{i} / {C}_{0 , i}}} , \qquad ( 3 )$$

In Equation 3, i denotes the uncleaved (=ucl) or cleaved (=cl) data, is the (background and flatfield) corrected fluorescence, m is the slope of the linear regime of the cleaved or uncleaved reporter calibration curve, c is the reporter concentration, and C₀ is the value that is varied to obtain
the best fit. Once C_{0,ucl} and C_{0,cl} were obtained, the conversion of fluorescence values to molar concentration of cleaved reporters followed the equation

$${c}_{cl} = \frac{F {10}^{\frac{{c}_{0}^{-}}{{C}_{1}}} - {F}_{ucl} {c}_{0}}{{F}_{cl} - {F}_{ucl}} , \qquad ( 4 )$$

$${C}_{1} = \frac{{C}_{0 , ucl} + {C}_{0 , cl}}{2} , \qquad ( 5 )$$

In Equation 4, F is the measured fluorescence during the trans-cleavage experiment, F_{ucl} is the slope of the linear regime of the uncleaved fluorescence intensity versus reporter concentration best fit line, F_{cl} is the slope of the linear regime of the cleaved fluorescence intensity versus reporter concentration best fit line, and c₀ is the total reporter concentration for each experiment.

The initial reaction velocities (in nM/s) for Michaelis-Menten analyses were obtained using a linear fit to the first ˜300 s of the trans-cleavage progress curves. The reaction velocities versus reporter concentration data were fitted to the Michaelis-Menten equation using GraphPad Prism 9 (GraphPad Software, CA, USA) to obtain Vₘₐₓ and K_{M}.

### Results

### Fluorescence-Based Detection of SNPs

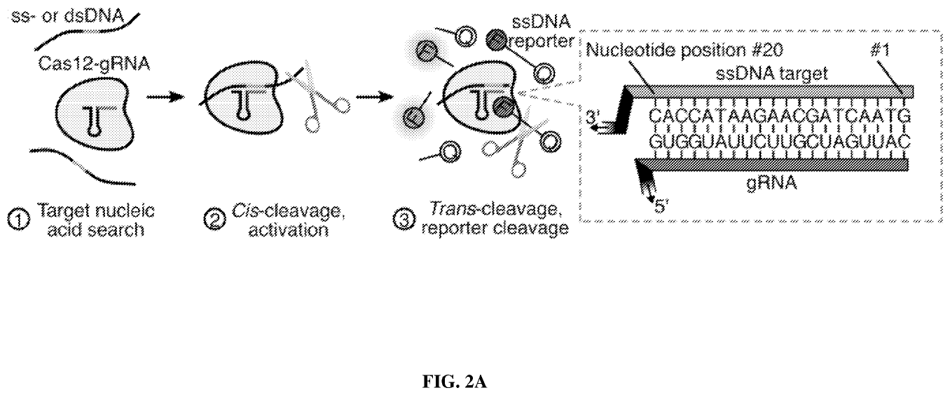

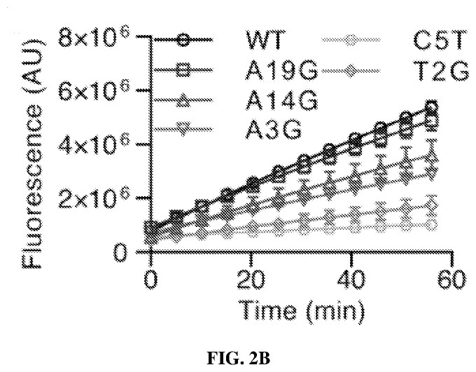

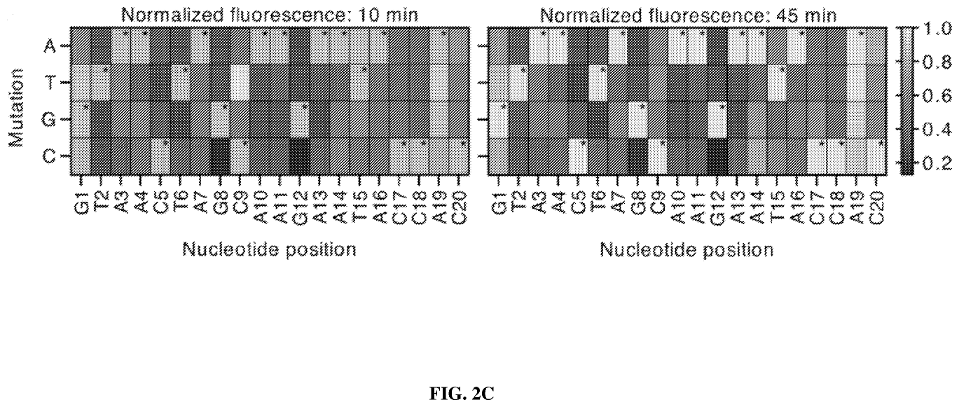

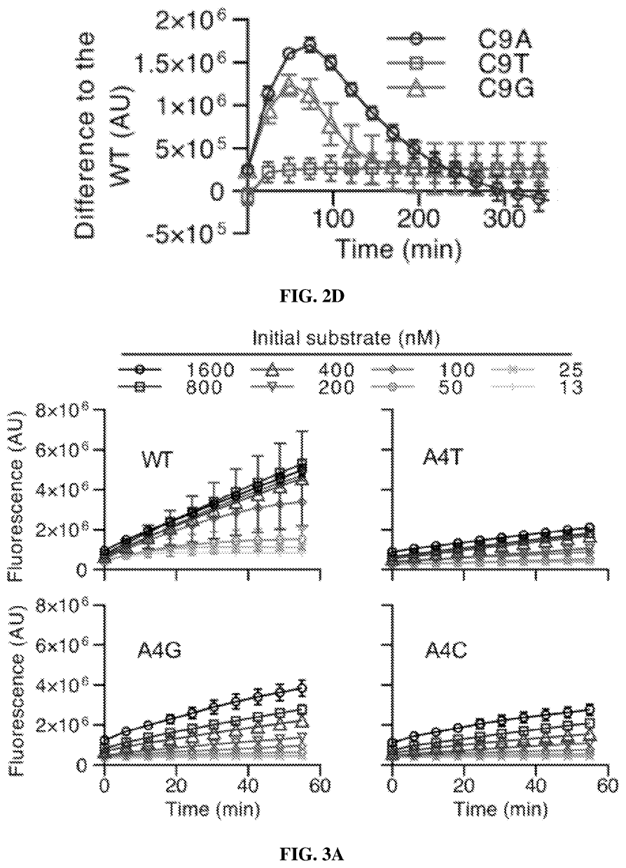

Progress curves were measured for the trans-cleavage reaction LbCas12a of the WT and each of the 60 target SNP species. (Methods for complete experimental protocol). The purpose of these measurements was to assess the ability of simple endpoint fluorescence assays to differentiate among these mutants. LbCas12a was complexed with a gRNA and then introduced a complementary WT (or almost complementary SNP ssDNA) target to activate the complexes (FIG. 2A). As a control, the same concentration of WT and mutant target across all experiments was used. The incubation of the Cas-gRNA complex with WT and SNP targets initiated trans-cleavage of fluorescently labeled ssDNA reporters (FIG. 2B). The SNPs are denoted using a three-parameter code (e.g., T2G indicates that the nucleotide position #2 contains the mutation wherein thymine (T) is replaced by guanine (G)). Heat maps of endpoint fluorescence at 10 and 45 min (FIG. 2C) revealed strong differences in the trans-cleavage activity among WT and SNPs. Also, in all heat maps, the WT is indicated with an asterisk in the top right corner of the corresponding cell.

These experiments show that the degree to which endpoint-fluorescence measurements enable differentiation of mutants depends strongly on the time at which the fluorescence was measured. For example, mutant C9T, showed greater fluorescence than WT at 10 min (FIG. 2C, left heat map), but significantly less fluorescence than WT at 45 min (FIG. 2C, right heat map). To better understand this behavior, the difference was measured in fluorescence signal between WT and SNPs in position #9 (C9T, C9A, and C9G) for longer times and at 5-fold higher target concentrations (FIG. 2D). C9T initially cleaved faster than WT (i.e. its value in FIG. 2D is initially negative) but the C9T signal was then overtaken by that of the WT after about 5 min. This behavior is consistent and can be explained by the underlying kinetics. This is demonstrated using experimentally validated Michaelis-Menten model informed by values of K_{M} and k*_{cat}/K_{M} measured here. (FIG. 8 and discussion). FIG. 2D shows how the WT-to-mutant fluorescence difference for all mutants fluctuate but then plateau near zero, as expected.

These results suggest that endpoint fluorescence is not well suited to differentiate between WT from SNPs primarily due to three reasons. First, endpoint fluorescence values depend on the differing amounts of (unmeasured) enzyme activity prior to fluorescence measurements (such as during pipetting or loading). Second, endpoint fluorescence values necessarily grow from zero and then saturate to approximately the same value as all reporters are cleaved. Differential trans-cleavage rates therefore mean that the choice of measurement time for endpoint detection is critical and depends on the reporter concentration. Third, the rate of fluorescence increase is largely determined by the (often unknown) concentration of activated enzymes. Hence, a SNP which exhibits a low trans-cleavage rate could nevertheless lead to larger fluorescence increase (relative to WT) when the SNP target is in sufficiently higher concentration. These limitations of endpoint-based detection can be mitigated by characterization of the underlying Michaelis-Menten kinetics. Namely, quantification of K_{M} and k*_{cat}/K_{M} provides a framework to address irregularities during sample preparation, choice of assay time, and unknown target concentrations. Further, quantification of K_{M} and k*_{cat}/K_{M} results in significantly greater specificity of detection.

### Effect of SNPs on Apparent Turnover Rate

To characterize the underlying Michaelis-Menten kinetics of SNP-activated trans-cleavage activity, apparent turnover rates (k*_{cat}) were measured. This value is defined by interpreting the data as if all Cas12-gRNA complexes are trans-activated by WT or SNP targets.

That is, k*_{cat} is defined as the ratio of the maximum achievable reaction velocity divided by the target concentration. k*_{cat} (rather the underlying turnover rate k_{cat}) is reported because the fraction of activated Cas12-gRNA complex is unknown for the SNP targets studied here, particular for complexes of mutants. Furthermore, the Cas12-gRNA complex and target concentrations were fixed across experiments to quantify comparable values of k*_{cat}.

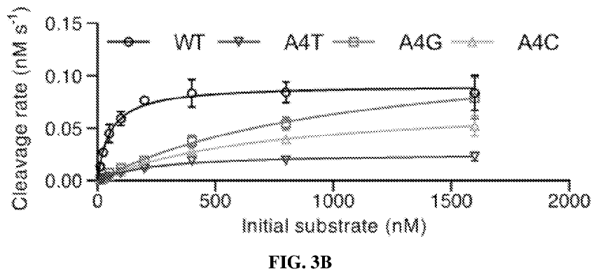

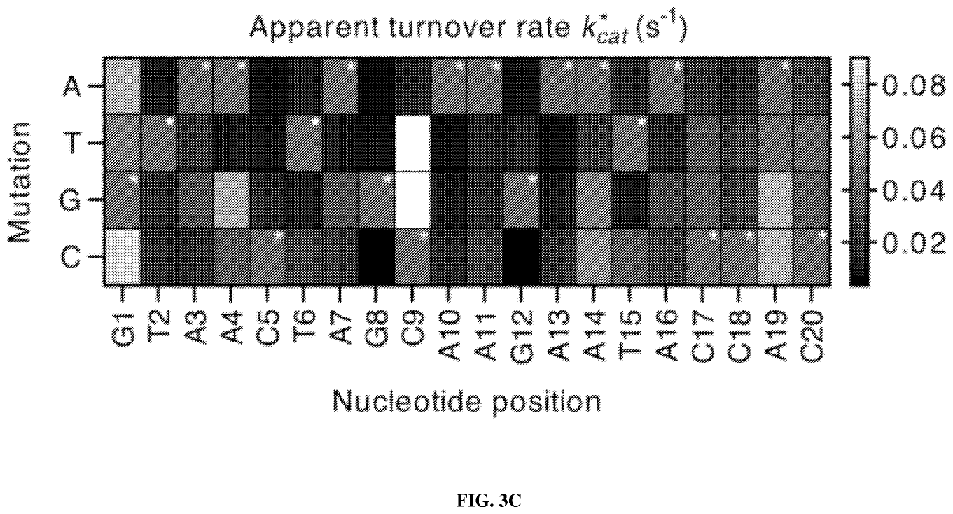

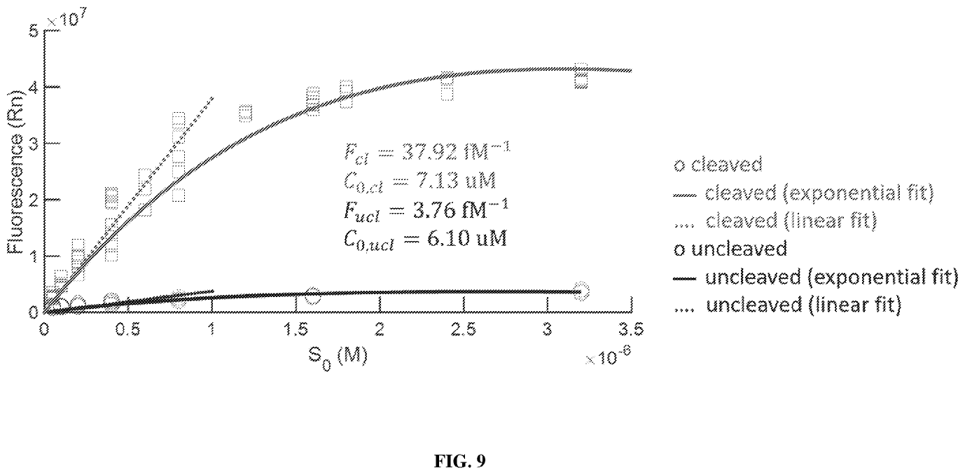

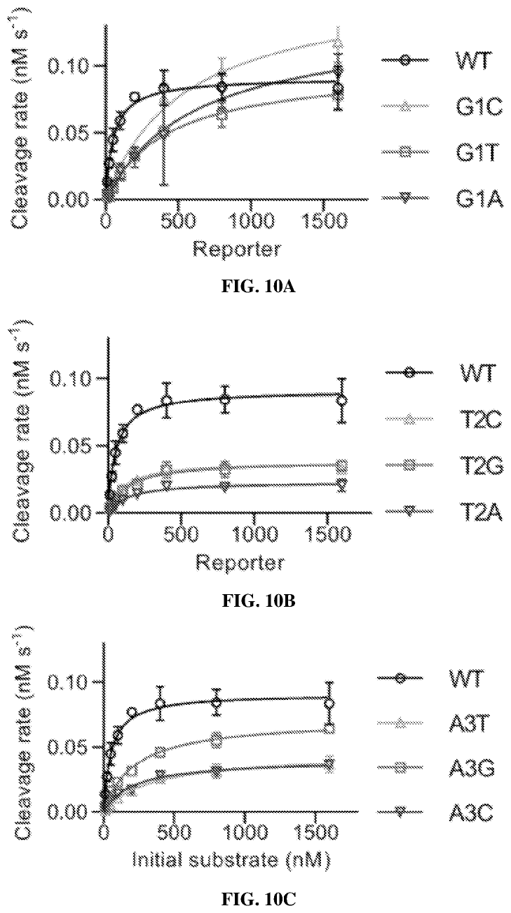

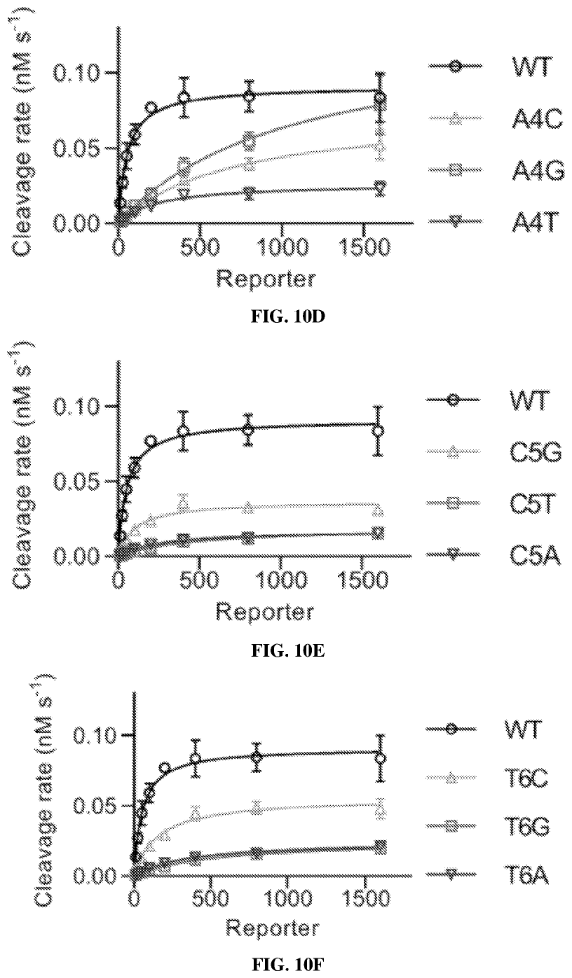

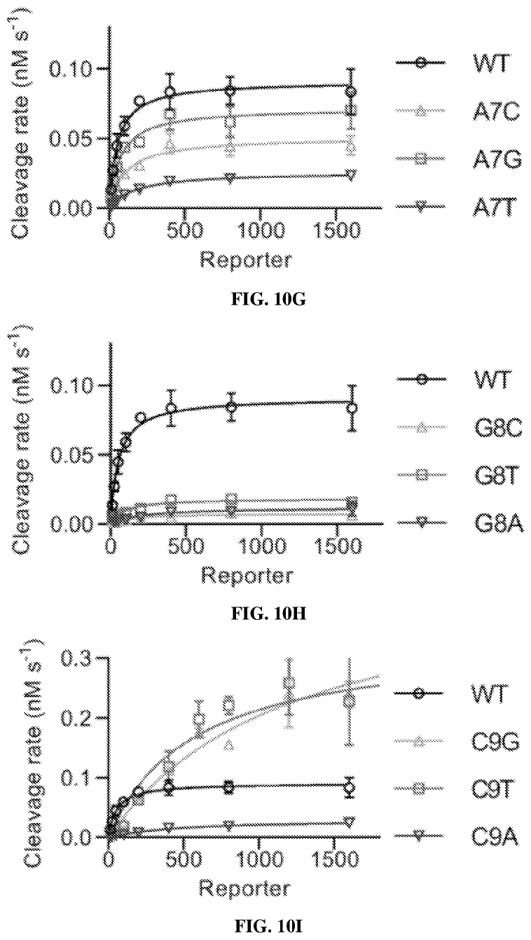

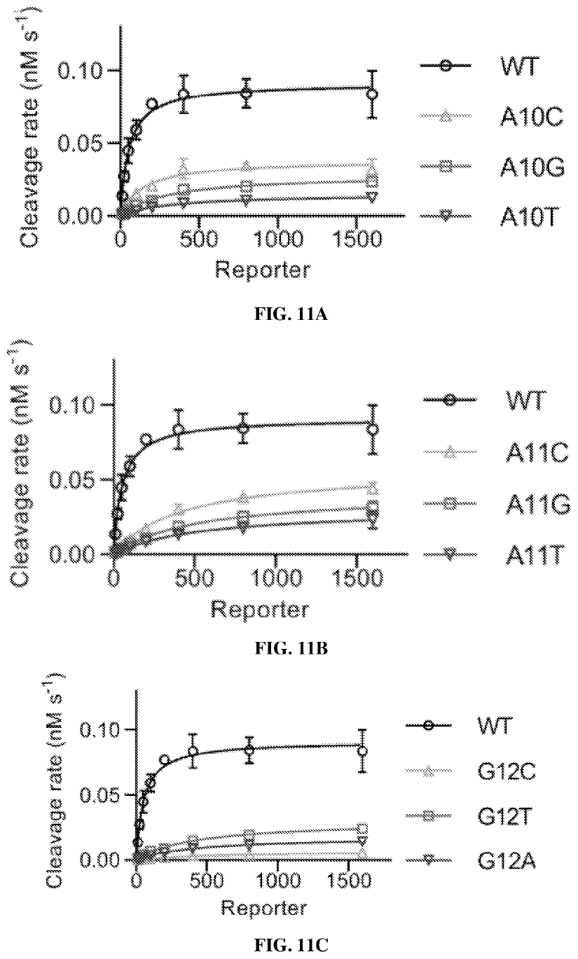

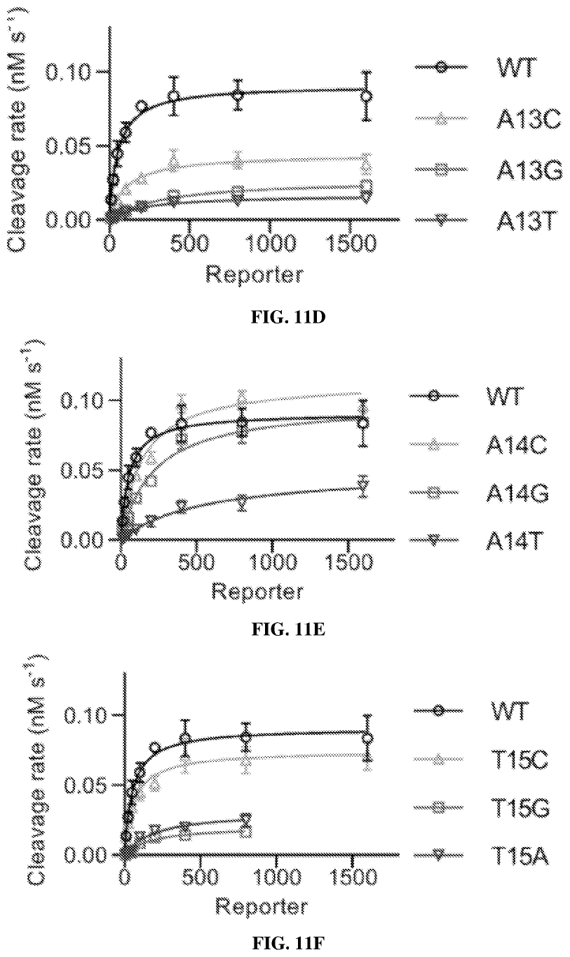

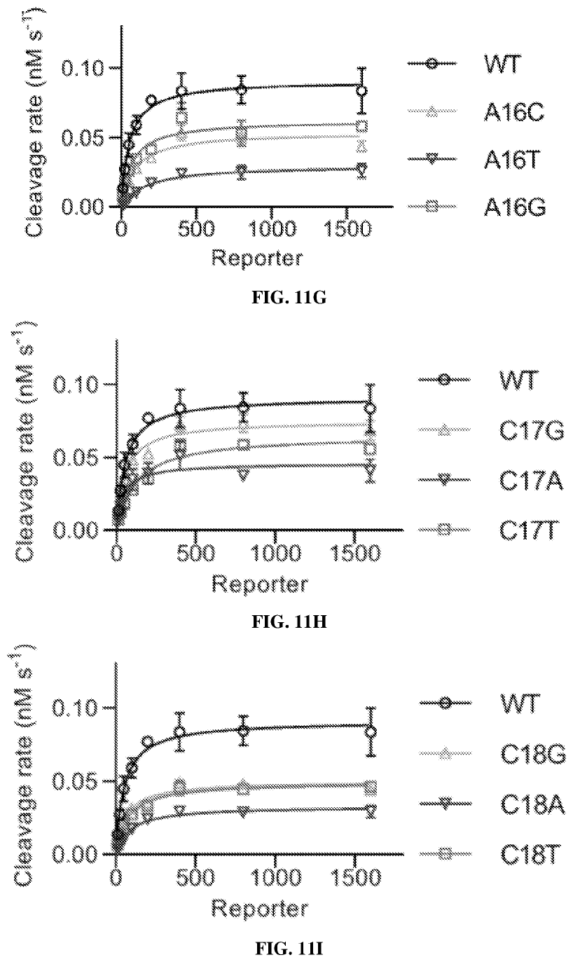

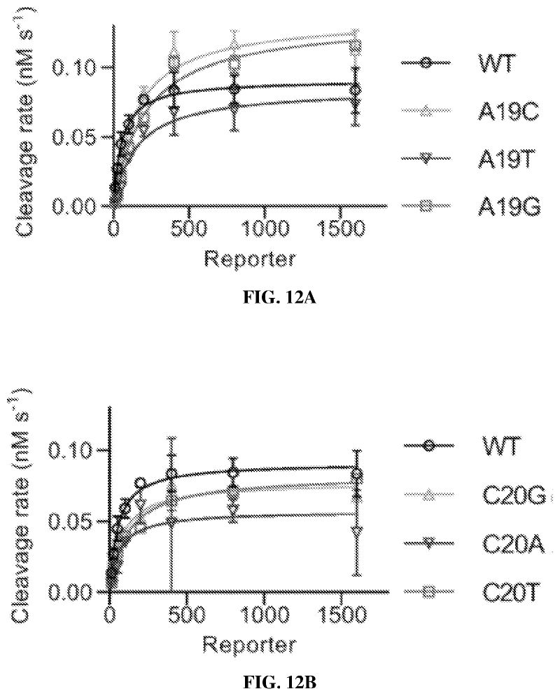

To that end, trans-cleavage progress curves for WT and SNP targets was measured with varied reporter concentrations between 13 and 1600 nM (example plots for position #4 are shown in FIG. 3A). The initial reaction velocities for each reactant system were then fit to a Michaelis-Menten curve (FIGS. 3B and 9-12). The Michaelis-Menten curve, importantly, yielded estimates of the target-associated k*_{cat} for these and all other SNPs studied here (FIG. 3C). [00152]k*_{cat} varied widely over the SNPs. For example, the WT k*_{cat} (=0.046/s) was surprisingly lower than that of 8 of the 60 SNPs (G1A, G1C, A4G, C9T, C9G, A14T, A19G, and A19C). In fact, the largest k*_{cat} was observed for C9G (4.9-fold greater than the WT k*_{cat}). These results suggest that single nucleotide mutations can significantly affect the trans-cleavage behavior of Cas12 enzymes in a manner which can result in both increases and decreases in apparent turnover rates. For differentiation among WT and SNP targets, measurements of k*_{cat} offer a technique that is insensitive to the duration of the experiment (since k*_{cat} is a fundamental property of the reactant system). Unfortunately, k*_{cat} typically requires a priori knowledge of the activated enzyme concentration. In turn, the latter can be deduced from the Cas12-gRNA complex concentration or the concentration of the target (whichever is lower and therefore limiting).

### Effects of SNPs on Michaelis-Menten Constant

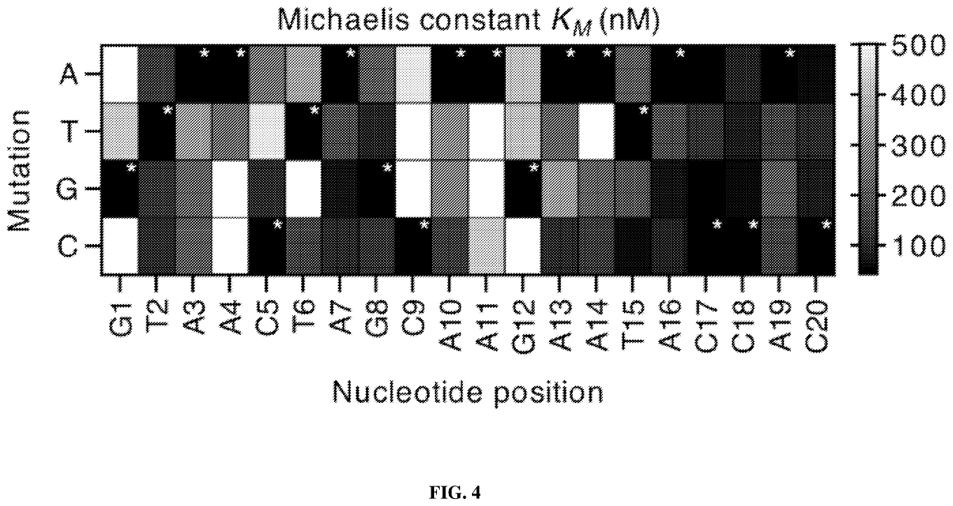

To detect WT in a manner independent of experiment duration and a priori knowledge of target concentration, the K_{M} was used. K_{M} describes the fundamental affinity of the enzyme for substrates and can be thought of as a property unique to a specific combination of Cas12-gRNA, target, buffer chemistry, and temperature. K_{M} also varied widely across SNPs (FIGS. 4 and 10-12). However, importantly, K_{M} offered much improved differentiation of SNPs versus WT. All but one SNP studied here (C17A, =43 nM) had higher K_{M} values (lower affinity) than the WT (=53 nM). These two values were also well within measurement uncertainty (Table 1). Surprisingly, the nucleotide positions (positions #18 to 20) had K_{M} values closer to the WT, suggesting a deterministic relation between SNP position and affinity. Interestingly, 6 of the 8 SNPs with a higher k*_{cat} than WT, showed K_{M} above 500 nM (almost 10-fold higher than WT). Further, the highest K_{M} was found for SNP C9G (1200 nM).

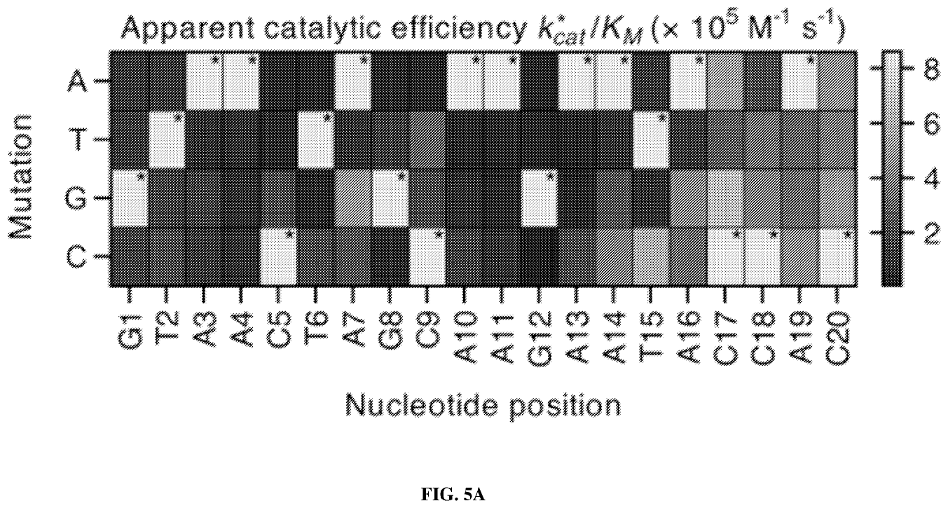

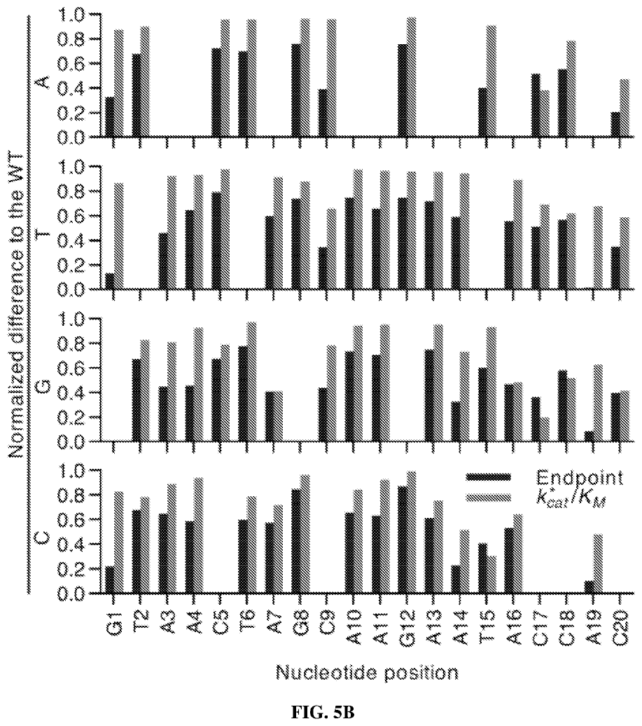

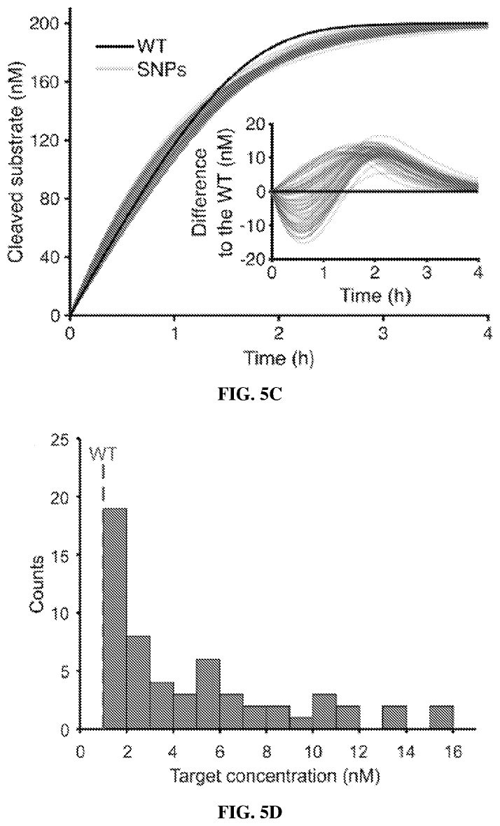

The K_{M} study showed that the WT-activated enzyme complex has a much stronger affinity to the trans-cleavage substrates (ssDNA reporters) compared to its SNP-activated counterparts. This strongly suggests that K_{M} can be a robust method to distinguish WT from SNPs. Most striking is that K_{M} is a fundamental property of the reactant system which enables WT differentiation without the requirement of a specified assay time or knowledge of target concentration. Instead, K_{M} assay directly measures the affinity of differently activated enzymes (WT or mutant) to substrates. Thus, this Examples describes the design of highly specific CRISPR-based, trans-cleavage assays for SNP detection. Following the determination of both k*_{cat} and K_{M} (both parameters derived from the same experiment), the apparent catalytic efficiency was calculated as k*_{cat}/K_{M}. A comparison of the 60 SNPs to the WT shows that k*_{cat}/K_{M} is a highly specific recognition of WT target (FIG. 5A). In fact, none of the 60 SNPs yielded a higher k*_{cat}/K_{M} than the WT. Further, the WT k*_{cat}/K_{M} (8.6×10⁵ M-1 s-1) was 130-fold higher than the lowest SNP k*_{cat}/K_{M}. The latter can be compared to the modest 7.8-, 13-, and 23-fold differences observed using endpoint fluorescence after 45 min for k*_{cat} and K_{M}. In addition to its exceedingly high specificity, quantification of k*_{cat}/K_{M} could potentially be automated and parallel measurements at various dilutions can potentially be faster than endpoint fluorescence experiments since the relevant initial cleavage rates can be measured within about 5 min. A limitation of this assay approach is, of course, that it requires knowledge of the target concentration.

As in the case of the K_{M} data, SNPs at nucleotide positions (#17 to 20) had k*_{cat}/K_{M} values closer to that of the WT. To more closely study the effect of SNP position on either endpoint- or catalytic efficiency-based differentiation of WT, their normalized difference to the WT was calculated (FIG. 5B). This difference was termed as D, which was calculated mathematically according to the following equation.

$${D}_{i} = 1 - \frac{{S}_{SNP}^{i}}{{S}_{WT}^{i}} \qquad ( 1 )$$

In Equation 1, the superscript i denotes detection by endpoint fluorescence after 45 min (i=EF) or k*_{cat}/K_{M} (i=CE), S is the relevant signal and the subscript SNP corresponds to a particular SNP. For example, S^{EP}_{C9T} corresponds to the endpoint fluorescence of SNP C9T while S^{CE}_{WT} corresponds to k*_{cat}/K_{M} of the WT. DCE was greater than DEF for all associated SNPs except T15C, C17A, C17G, and C18G. For a fixed mutation base (say, mutations to A), DCE mostly decreased with increasing nucleotide position while DEF was greatest towards the middle nucleotide positions.

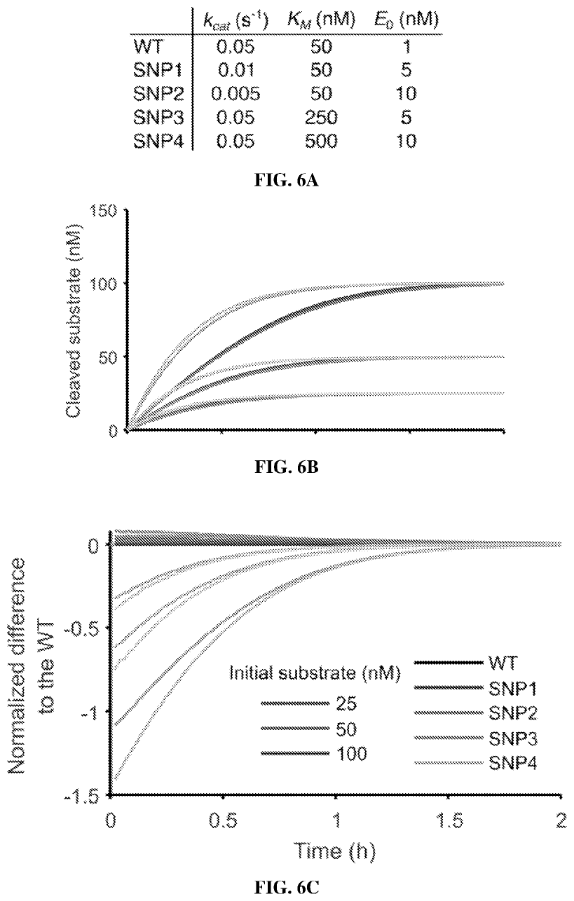

Experimentally validated Michaelis-Menten model was used to map out the scenarios wherein K_{M} and k*_{cat}/K_{M} were the ideal variables to differentiate WT from SNP targets. Now, the endpoint detection timescale was proportional to the quantity K_{M}/k*_{cat}E₀(where E₀ is the activated enzyme concentration). Therefore, conditions (either WT or SNP) that result in equal K_{M}/k*_{cat}E₀ would yield approximately equal endpoint fluorescence. To test this hypothesis, the cleaved substrate concentration versus time was predicted for four different contrived (hypothetical) samples (FIG. 6A). Samples with lower K_{M} cleaved faster than WT (due to the correspondingly high E₀ (FIGS. 6B and 6C). Interestingly, this difference became greater with increasing initial reporter concentration.

In conclusion, fundamental Michaelis-Menten parameters are proposed as a time- and target concentration-insensitive modality for SNP differentiation. The proposed method yielded greater dynamic range than endpoint fluorescence and provides faster detection (since initial reaction velocities can be measured within 5 min). Also, although results for “pure” mutant samples (i.e., mutants that are not mixed with WT) are presented, a mixture of WT and C9A gave Michalis-Menten kinetics closer to the WT (Table 2.)

TABLE 2
List of gRNAs used in this work.

| Name | Type | Sequence (5′-3′) |
|---|---|---|
| gRNA | ssRNA | UAA UUU CUA CUA AGU |
| | | GUA GAU GUG GUA UUC |
| | | UUG CUA GUU AC (SEQ |
| | | ID NO: 1) |
| WT (All SNPs | ssDNA | G TAA CTA GCA AGA |
| are generated | | ATA CCA C (SEQ ID |
| based on this | | NO: 2) |
| WT sequence) | | |
| Rep | ssDNA | [5-Fam] TTA TTA TT |
| | | [3-BHQ-1] |

### Example 3—CRISPR Kinetics Assays for Specific, Robust, and Deployable Genotyping

The number of druggable tumor-specific molecular aberrations has grown substantially in the past decade, with a significant survival benefit obtained from biomarker matching therapies in several cancer types. Tumor markers are not limited to therapies. They also help screen a healthy population or a high-risk population for the presence or development of cancer. Certain tumor markers facilitate early diagnosis of a specific type of cancer and even determine prognosis for a patient. Certain other tumor markers help monitor patients in remission or while having surgery, radiation, or chemotherapy.

While sequencing may be used for detecting tumor markers, despite the sequencing revolution, genotyping is still not broadly available, neither for early BRCA risk assessment nor for non-small cell lung cancer tumor (NSCLC) genotyping. Lung cancer remains the number one cause of cancer-related deaths. For its most common form (NSCLC claims 85% of cases), EGFR is the first metastatic driver gene with 30% prevalence. However, 20-30% of patients with newly diagnosed NSCLC do not have EGFR genotyping prior to initiating therapy, leading to inadequate treatment for EGFR targeted therapies. As for BRCA testing, only 20% of eligible US women have accessed/undergone genetic testing and more than 80% of breast cancers are diagnosed at Stage II or greater in low- and middle-income countries decreasing survival rates. These data highlight the unmet clinical need for the extension of early BRCA screening and EGFR tumor genotyping in-house with low-equipment, particularly, in populations with limited resources.

NGS, the gold standard for genotyping, is currently performed through amplification followed by the amplicon screening. This allows high sensitivity but has many drawbacks, notably cost of reagents and equipment, workflow complexity, and amplification bias. These factors make NGS unsuitable to low-resource settings.

Existing CRISPR-based assays leverage programmable guide RNAs complexed with CRISPR to identify DNA/RNA mutations. However, differentiation among mutants has been limited to fluorescence-based assays. Also, these assays require prior knowledge of mutant concentration and specific endpoint time which has limited their applications.

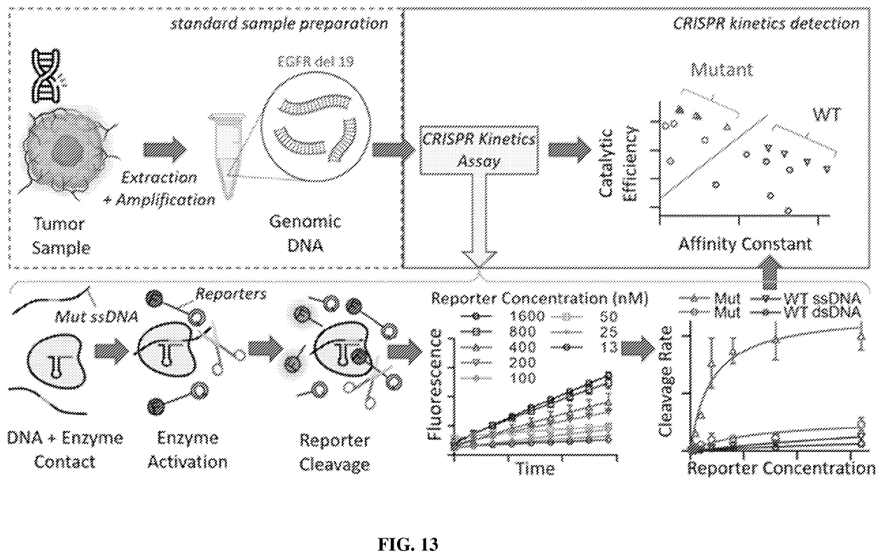

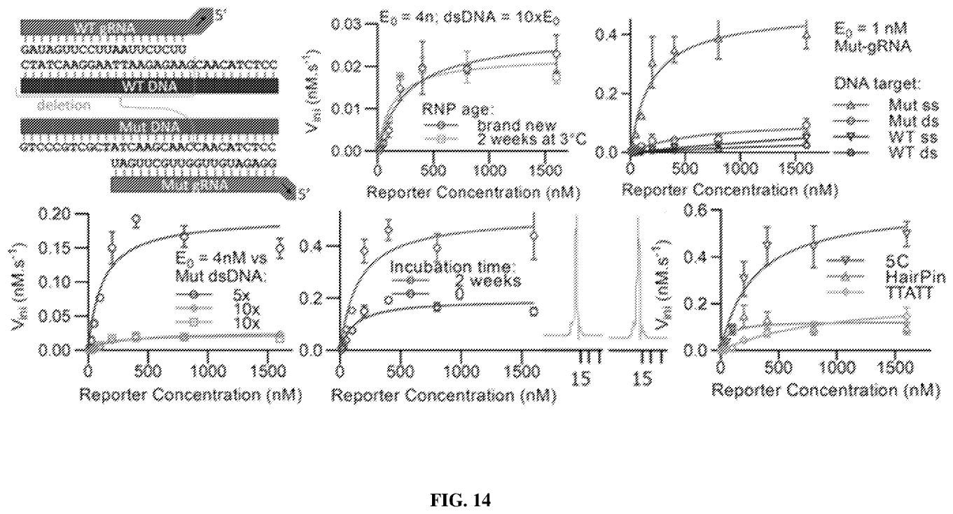

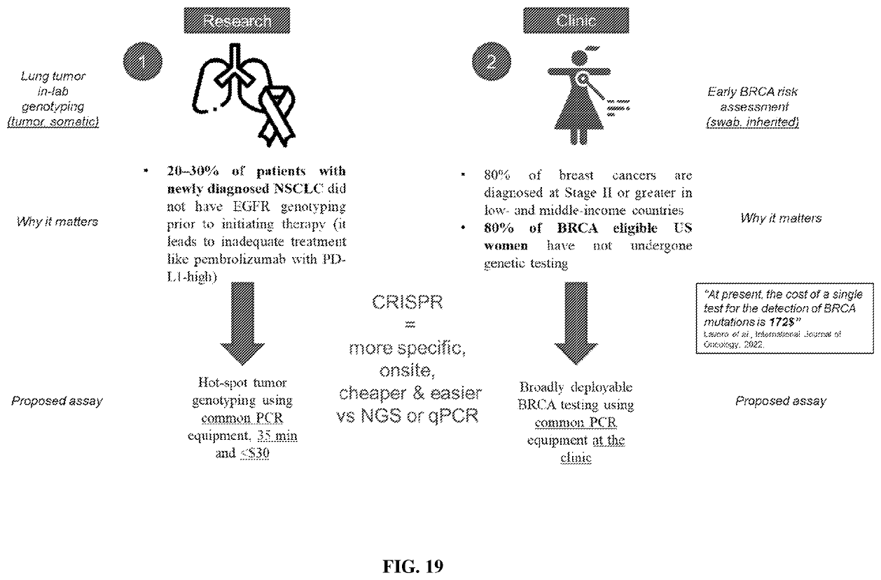

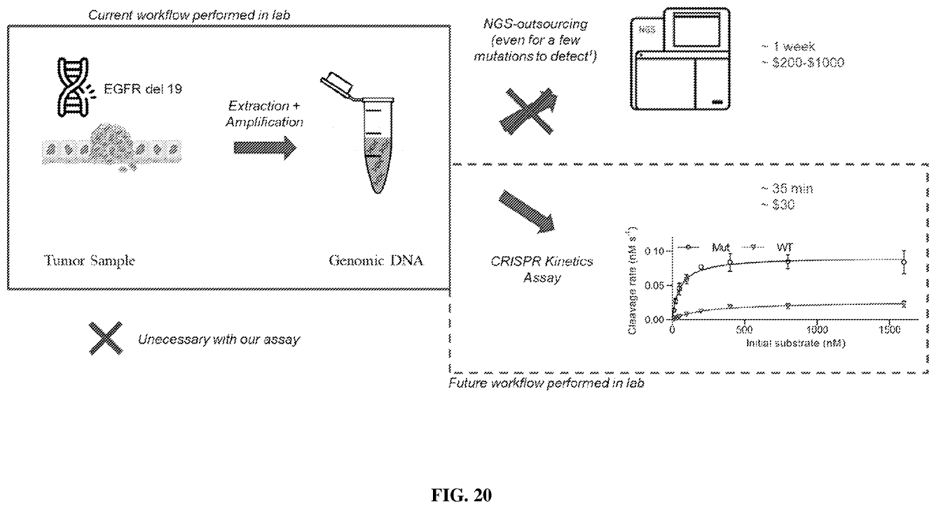

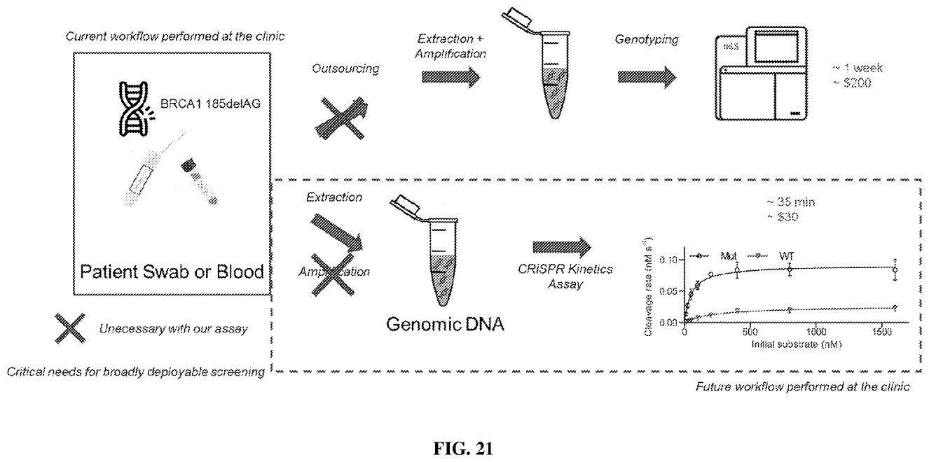

In contrast, CRISPR-based assays disclosed herein provide tumor marker detection with high specificity, low cost, and ease of use. These assays are independent of target concentration and time, making it robust and promising for real-world flexible genotyping in lab and genotyping broad screening for inherited genetic conditions. (FIGS. 19-21.) This Example describes one such assay for analyzing tumor marker referenced herein as “EGFRdel19” as well as screening of inherited pathogenic cancer mutations directly from a swab. A schematic representation of an example of such assay is provided in FIG. 13. FIG. 14 describes certain aspects of assay development and assay components that may influence CRISPR endonuclease kinetics and result analysis.

Certain aspects of the assay design are summarized below:

gRNA Design: WT and mutant sequence choice: gRNA is designed such that the mismatch is central on the gRNA to enhance specificity for the target DNA. WT vs mutant complexation: the use of Mu-gRNA for assay to avoid false positive and WT-gRNA for assays to avoid false negative. The CRISPR-gRNA complex is very specific to the target DNA. Hence, the guide RNA design is a flexible tool to adapt the assay to meet either very high sensitivity requirements (Mu-gRNA to avoid false positive) or very high specificity requirement (WT-gRNA to avoid false negative).

The gRNA-CRISPR endonuclease complex or ribonucleoproteins (RNP) can be stored in a refrigerator, i.e., between 2° C. to 8° C. for weeks with minimal to no impact on activity.

DNA type study (ssDNA vs dsDNA). The RNP has, on average 5-fold less decrease in catalytic efficiency form ssDNA target to dsDNA target. It is an important factor to consider in the design of an assay targeted as small concentrations of DNA target without amplification. In this case, the RNP concentration matches the target concentration. Without amplification, a denaturation step (transforms dsDNA in ssDNA) with 60% DMSO after DNA target extraction will increase the limit of target concentration detectable by a factor on average 5. With amplification, the target is in excess of the RNP, the limit of detection is no longer a problem as RNP concentration can be increased to increase signal.

Longer incubation in the refrigerator after in-excess-of-target-activation improves catalytic efficiency, i.e., the more duration of the storage, the higher is the CRISPR activity. CRISPR activity is indeed observed on DNA target even when the target is competing against reporters. Therefore, there is an optimum concentration of target to reach the maximum apparent catalytic efficiency. Too low concentration of DNA target decreases the amount of activated enzyme, hence apparent catalytic efficiency. Too high concentration of DNA target competes with reporter cleavage and decreases apparent catalytic efficiency.

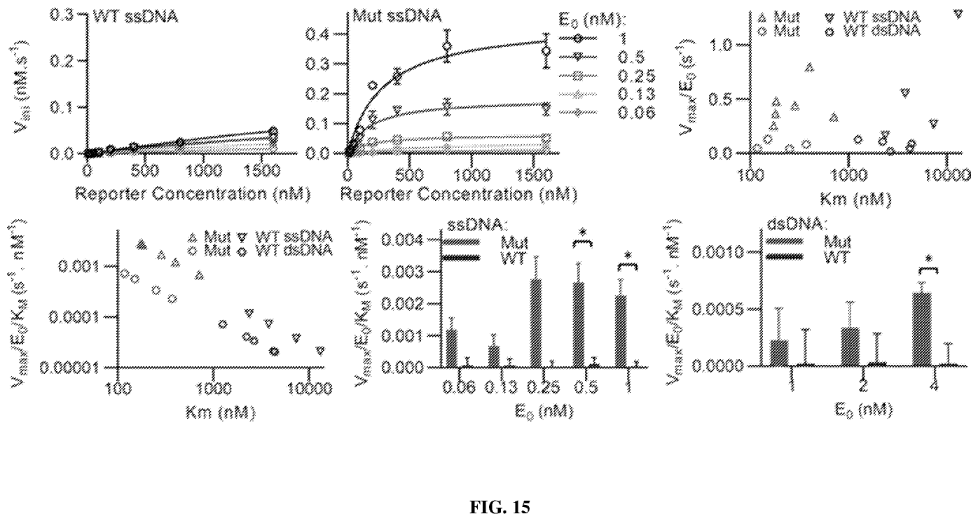

Synthetic sensitivity study was performed for ssDNA and dsDNA, as shown in FIG. 15. Catalytic efficiency was the best parameter to assess sensitivity. Therefore, the lowest E0 can be determined that differentiates between WT and Mutant using catalytic efficiencies. (FIG. 15.)

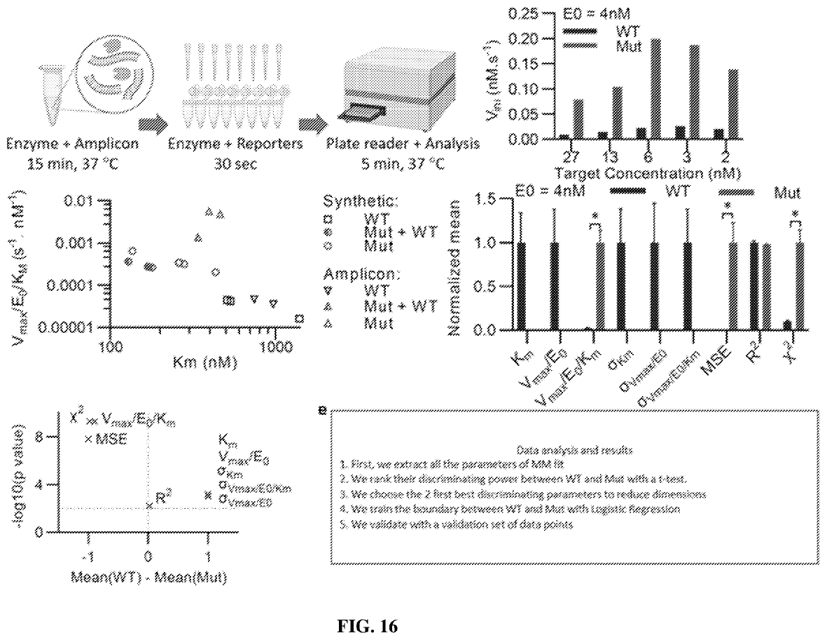

An exemplary work-flow for tumor genotyping for a tumor marker is provided in FIG. 16. CRISPR endonuclease and target nucleic acids, for example, amplified genomic target nucleic acids can be incubated for between 10 minutes and 20 minutes, particularly, for 15 minutes, at a temperature between 35° C. and 40° C., particularly, at 37° C. The mixture can then be mixed with reporters at different concentrations for between 15 seconds and 60 seconds, particularly, for 30 seconds. The reaction mixture is then read in a plate reader for between 3 minutes and 10 minutes, particularly, for about 5 minutes, at a temperature between 35° C. and 40° C., particularly, at 37° C.

Different kinetic parameters were then analyzed to determine whether the target nucleic acid contains a WT or a mutant nucleic acid.

For example, all the parameters were extracted to describe the Michaelis Menten fit as well as parameters with a physical meaning like k_{cat}, K_{M}, k_{cat}/K_{M}, and goodness of fit. The power of these parameters to differentiate between WT and mutant with a t-test was ranked. The first two best discriminating parameters (that is k_{cat}/K_{M} and Rsquare) were chosen to reduce the dimension to 2 parameters and increase robustness of the predictive model. The data boundary was then trained between WT and mutant with Logistic Regression. Such analysis was validated with a validation set of data points.

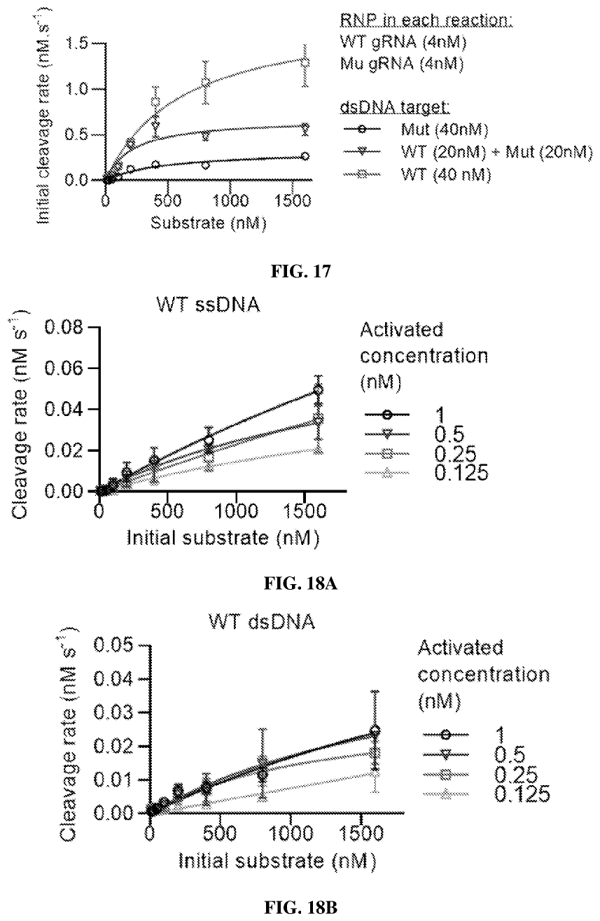

Multiplexing CRISPR observations: The reactions containing the mixture of WT and mutant target nucleic acids did not show the highest initial velocities, even though velocities of both perfectly activated RNPs should add up. (FIG. 17.) Thus, K_{M} is not additive, if we consider activation was a first order reaction, i.e., fully activated RNPs when targets are in excess. Rather, the activation is an equilibrium that can be displaced by adding a target. Indeed, it explains why the reactions containing WT and mutant nucleic acids do not show the highest velocities.

In other words, if activation was a first order reaction, the reactions containing a mixture of WT and mutant nucleic acids should be the addition of WT curve and mutant curve. However, the results suggest that WT+Mut curve=½ WT curve+½ Mut curve. This can be due to target competition with reporters.

Further, FIGS. 18A and 18B show that similar results were obtained with single stranded WT and mutant nucleic acids. These data show that 100 pM sensitivity can be obtained for early inherited cancer risk screening. Also, denaturation of genomic dsDNA to ssDNA may be performed with 60% DMSO.

### Example 3—Diagnostic Assays for WT and SNP Detection

This example proposes further assay optimization, performance quantification, and demonstration of two novel assays to maximize CRISPR diagnostics specificity. FIGS. 7A and 7B, respectively, depict the arbitrarily names Assay 1 and Assay 2.

Assay 1 in FIG. 7A begins with either an unamplified or amplified target nucleic acids with or without some standard, nonquantitative pre-amplification (e.g. polymerase chain reaction, PCR, or loop-mediated isothermal amplification, LAMP). Importantly, the assay results are independent of the original sample concentration since knowledge of the chemical kinetics was used to extract properties specific to the reacting species. This amplification can all be performed with the same (existing) WT-specific primers. Given this input, 8 to 10 dilutions of the reporter can be performed and a standard thermocycler can be used to obtain 24 to 30 simultaneous progress curves in a single 5 min run. Only about 5 min would be needed since the method is independent of signal strength and only the signal rates of change versus dilution are important. These data could be used to build a Michaelis-Menten curve, extract K_{M} using existing algorithms, and identify the sample as either WT or mutant with single-base specificity. Mutant samples flagged in this manner can then be referred to other analyses, including more expensive sequencing.

Assay 2 in FIG. 7B begins with a raw sample (e.g. sample extraction from pharyngeal swab solution), of possibly unknown concentration. Two possible paths would then be evaluated. The lower path would use a standard non-quantitative amplification followed by a measurement of amplicon concentration. The latter can be performed with a standard spectrofluorometer or be integrated with the amplification (e.g. real-time PCR). Next, the amplicon of known concentration can be processed as per the protocol of Assay 1. Lastly, algorithms could be used to extract K_{M} and k*_{cat}/K_{M} for an increased specificity. An even more specific classification (as WT or mutant) can then be performed.

In FIG. 7B, the box labeled “Novel amplification” may not be required for the proposed assays. Amplification master mixes (e.g. for LAMP) could be designed with a precisely limited reagent to produce amplicons of known concentrations (or at least known to within about a factor of 2.). This would enable very high specificity and reduced cost and time by obviating the need to quantify sample concentration in Assay 2.

In certain cases, a neural network could be trained to extract kinetic rate parameters from the progress curves (basically raw data with minimal scaling to account for calibration) resulting multiple dilution experiments performed in parallel instead of traditional Michaelis-Menten kinetics assays.

Much of the initial training can be performed for such a method using simulations of the enzyme kinetics with superposed (artificial) noise on the data. Experimental data can then be trained. A second variation would be to use artificial intelligence (or traditional cluster analyses) to identify individual strains and/or mutations in the sample given kinetic parameters such as extracted K_{M} and k*_{cat}/K_{M}. The latter may be possible but will require much more data and training of a neural network. Thus, Assay 2 would offer higher sensitivity since it would include pre-amplification and the maximum specificity since both K_{M} and k*_{cat}/K_{M} would be quantified.

### Example 4—Fulfilling Unmet Clinical Needs

The methods disclosed herein find a variety of applications to extremely high specificity and low-equipment detection of genetic markers. One obvious application is to monitor SNPs, variants, and/or subvariants of pathogens in infectious diseases such as SARS-CoV-212 and HIV. For example, the assays disclosed herein could be performed economically on either PCR or LAMP amplicon of samples which test positive.

A second example would be the screening of genetic cancer markers for detection of cancer risks without amplification and for precise monitoring of gene therapies including detection of up- or down-regulated target genes. This latter area is one where CRISPR diagnostics is just beginning to have an impact. For example, one test using CRISPR-Cas13 is proposed by Palaz et al., ACS Synth. Biol. 10, 1245-1267 (2021). By comparison, the assays proposed herein would be target concentration independent, more robust, and faster with no need for amplification.

Although the foregoing invention has been described in some detail by way of illustration and example for purposes of clarity of understanding, it is readily apparent to those of ordinary skill in the art considering the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. (canceled)

2. A method of comparing a sequence of a target nucleic acid with a sequence of a reference nucleic acid, comprising contacting the target nucleic acid as a reagent and a plurality of concentrations of a reporter as a substrate with a Clustered Regularly Interspaced Short Palindromic Repeats (CRISPR) endonuclease comprising a guide RNA (gRNA) that hybridizes with the reference nucleic acid, and quantifying an enzyme kinetic parameter of the CRISPR endonuclease.

3. The method of claim 2, wherein the enzyme kinetic parameter comprises the rates of cleavage of the reporter at different concentrations of the reporter, optionally wherein the rates of cleavage are initial rates 1 to 10 minutes after initiation of reporter cleavage.

4. (canceled)

5. (canceled)

6. The method of claim 3, wherein the enzyme kinetic parameter is selected from the group consisting of a Michaelis-Menten constant (K_{M}) of the CRISPR endonuclease, an apparent enzyme turnover rate (k*_{cat}) of the CRISPR endonuclease, an enzyme turnover rate (k_{cat}) of the CRISPR endonuclease, apparent catalytic efficiency (k*_{cat}/K_{M}) and catalytic efficiency (k_{cat}/K_{M}).

7-10. (canceled)

11. The method of claim 2, wherein the target nucleic acid is amplified using an enzymatic exponential nucleic acid amplification before using the target nucleic acid amplicon as the reagent in assaying the enzyme kinetic parameter of the CRISPR endonuclease.

12. The method of claim 2, wherein analyzing the target nucleic acid comprises identifying the target nucleic acid as having the same sequence as the reference nucleic acid or a different sequence from the reference nucleic acid.

13. (canceled)

14. The method of claim 2, wherein assaying the enzyme kinetic parameter of the CRISPR endonuclease comprises assaying enzyme activity of the CRISPR endonuclease (i) using the same concentration of the target nucleic acid at different concentrations of the reporter; or (ii) under conditions of excess amount of the target nucleic acid and/or excess amount of the reporter as compared to the amount of the CRISPR endonuclease.

15. (canceled)

16. A method of comparing a sequence of a target nucleic acid with a sequence of a reference nucleic acid, comprising contacting the target nucleic acid as a reagent and a plurality of concentrations of a reporter as a substrate with a Clustered Regularly Interspaced Short Palindromic Repeats (CRISPR) endonuclease comprising a guide RNA (gRNA) that hybridizes with the reference nucleic acid, and quantifying a rate[s] of cleavage of the reporter substrate at each of the plurality of concentrations of the reporter.

17. The method of claim 16, comprising quantifying an initial rate of cleavage of the reporter substrate at each of the plurality of concentrations of the reporter, optionally wherein the initial rate is the rate during the initial 1 to 10 minutes after initiation of the CRISPR endonuclease mediated cleavage of the reporter.

18. (canceled)

19. The method of claim 16, wherein a difference in the rates of cleavage by the CRISPR endonuclease of the reporter at the plurality of concentrations of the reporter in the presence of the target nucleic acid as compared to the rates of cleavage of the reporter by the CRISPR endonuclease at the plurality of concentrations of the reporter in the presence of the reference nucleic acid indicates that the sequence of the target nucleic acid is different from the sequence of the reference nucleic acid, and no difference in the rates of cleavage indicates that the sequence of the target nucleic acid is the same as the sequence of the reference nucleic acid.

20. (canceled)

21. The method of claim 16, wherein the plurality of concentrations of the reporter comprises a reporter concentration higher than the K_{M} of the CRISPR endonuclease and at least one reporter concentration is approximately equal to or smaller than the K_{M} of the CRISPR endonuclease.

22-25. (canceled)

26. The method of claim 2, wherein the reporter produces a detectable signal after being cleaved, optionally wherein the reporter comprises a fluorescence-emitting dye pair.

27-29. (canceled)

30. The method of claim 2, wherein the target nucleic acid is denatured from dsDNA to ssDNA before using the denatured target nucleic acid as a reagent in assaying the enzyme kinetic parameter of the CRISPR endonuclease.

31. A kit comprising:
a reporter molecule;
a reference nucleic acid;
a CRISPR endonuclease comprising a guide RNA comprising a sequence that is perfectly complementary to the reference nucleic acid; and
a non-transitory computer readable media comprising instructions, which when executed by a processor, cause the processor to analyze results of an assay of the CRISPR endonuclease using a target nucleic acid and the reporter molecule as a substrate and provide an enzyme kinetic parameter of the CRISPR endonuclease.

32-50. (canceled)
